(19) United States
(12) Patent Application Publication
(10) Pub. No.: US 2024/0216454 A1
(43) Pub. Date: Jul. 4, 2024

(54) METHOD FOR TREATING DERMATITIS AND PHARMACEUTICAL COMPOSITION FOR TREATING DERMATITIS CONTAINING AN ADENOSTEMMA LAVENIA EXTRACT

(71) Applicant: Industrial Technology Research Institute, Hsinchu (TW)

(72) Inventors: Kuo-Kuei HUANG, Zhubei City (TW); I-Hong PAN, Zhubei City (TW); I-Huang LU, Hsinchu City (TW); Shu-Fang WEN, Baoshan Township (TW)

(73) Assignee: Industrial Technology Research Institute, Hsinchu (TW)

(21) Appl. No.: 18/396,942

(22) Filed: Dec. 27, 2023

(30) Foreign Application Priority Data
Dec. 29, 2022 (TW) 111150610

(51) Int. Cl. A61K 36/28; A61P 17/00

(52) U.S. Cl. CPC A61K 36/28; A61P 17/00; A61K 2236/331; A61K 2236/37; A61K 2236/39; A61K 2236/51; A61K 2236/53

(57) ABSTRACT

A method for treating dermatitis is provided. The method for treating dermatitis includes administering an effective amount of an Adenostemma lavenia extract or administering an effective amount of a pharmaceutical composition for treating dermatitis including an Adenostemma lavenia extract to a subject in need thereof, wherein the dermatitis comprises atopic dermatitis or contact dermatitis.

### CROSS REFERENCE TO RELATED APPLICATION

The present application is based on, and claims priority from, Taiwan Application Serial Number 111150610, filed Dec. 29, 2022, the disclosure of which is hereby incorporated by reference herein in its entirety.

### TECHNICAL FIELD

The technical field relates to a method for treating dermatitis and a pharmaceutical composition for treating dermatitis containing an Adenostemma lavenia extract.

### BACKGROUND

Atopic dermatitis is one of the most common chronic diseases in the world. Based on data on the Global Burden of Disease shown by the World Health Organization (WHO), at least 230 million people worldwide suffer from atopic dermatitis and it is the number one cause of non-fatal skin disease burden.

Most of the external medicines for atopic dermatitis that are currently available have side effects. For example, use of steroids may cause side effects such as skin atrophy, striae atrophicae and capillary dilation. Therefore, natural medicines for dermatitis with little to no side effects are still the focus of urgent development.

### SUMMARY

The present disclosure provide a method for treating dermatitis, comprising administering an effective amount of an Adenostemma lavenia extract or administering an effective amount of a pharmaceutical composition for treating dermatitis including an Adenostemma lavenia extract to a subject in need thereof, wherein the dermatitis comprises atopic dermatitis or contact dermatitis.

The present disclosure also provides a pharmaceutical composition for treating dermatitis, comprising an Adenostemma lavenia extract and a pharmaceutically acceptable carrier or salt, wherein the dermatitis comprises atopic dermatitis or contact dermatitis.

A detailed description is given in the following embodiments with reference to the accompanying drawings.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The present invention can be more fully understood by reading the subsequent detailed description and examples with references made to the accompanying drawings, wherein:

FIG. 1 shows the hematoxylin-eosin staining results of the dorsal skin tissue sections of the mice in the group without sensitization treatment (Naïve), the group administered with the vehicle and the group administered with Adenostemma lavenia extract 0805 in the animal model experiment of 2,4-dinitrochlorobenzene (DNCB)-induced dermatitis. Position marked by an arrow indicates hyperplasia and position marked by * indicates inflammation.

FIG. 2 shows epidermal thickness of the dorsal skin of the mice in the group without sensitization treatment, the group administered with the vehicle and the group administered with Adenostemma lavenia extract 0805 in the animal model experiment of 2,4-dinitrochlorobenzene-induced dermatitis. Data are presented as mean±standard error (Mean±SEM) (n=5). Statistical analysis was performed by Student's t-test. Compared to the group administered with the vehicle, p<0.05 represents that there is a statistically significant difference, *p<0.05; ***p<0.001.

FIG. 3 shows total histological scores for the dorsal skin of the mice in the group without sensitization treatment, the group administered with the vehicle and the group administered with Adenostemma lavenia extract 0805 in the animal model experiment of 2,4-dinitrochlorobenzene-induced dermatitis. Data are presented as mean±standard error (n=5). Statistical analysis was performed by Mann-Whitney test. Compared to the group administered with the vehicle, p<0.05 represents that there is a statistically significant difference, *p<0.05; **p<0.01.

FIG. 4 shows photographs of mouse ears on the 30^{th} day of the experiment in the group without sensitization treatment, the group administered with the vehicle and the group administered with Adenostemma lavenia extract 0805-F and the group administered with cyclosporine A in the animal model experiment of 2,4-dinitrochlorobenzene-induced dermatitis.

FIG. 5 shows measurement results for mouse ear thickness on the 30^{th} day of the experiment in the group without sensitization treatment, the group administered with the vehicle, the group administered with Adenostemma lavenia extract 0805-F and the group administered with cyclosporine A in the animal model experiment of 2,4-dinitrochlorobenzene-induced dermatitis. Data are presented as mean±standard error (Mean±SEM) (n=5). For the comparisons of the ear thickness of respective groups to the ear thickness of the group administered with the vehicle, statistical analysis was performed by two-way ANOVA with Dunnett's multiple comparisons test. Compared to the group administered with the vehicle, p<0.05 represents that there is a statistically significant difference, ***p<0.001. For the comparisons of the ear thickness of the groups administered with 300 mg/kg Adenostemma lavenia extract 0805-F to the ear thickness of the group administered with cyclosporine A, statistical analysis was performed by Student's t-test. p<0.05 represents that there is a statistically significant difference, ###p<0.001.

FIG. 6 shows the hematoxylin-eosin staining results of ear skin tissue sections of mice in the group without sensitization treatment, the group administered with the vehicle, the group administered with Adenostemma lavenia extract 0805-F and the group administered with cyclosporine A. Position marked by an arrow indicates hyperplasia and position marked by * indicates inflammation.

FIG. 7 shows total histological scores for ear skin of mice in the group without sensitization treatment, the group administered with the vehicle, the group administered with Adenostemma lavenia extract 0805-F and the group administered with cyclosporine A. Data are presented as mean±standard error (Mean±SEM) (n=5). For the comparison of the total histological score of respective groups to the total histological score of the group administered with the vehicle, statistical analysis was performed by Mann-Whitney test. p<0.05 represents that there is a statistically significant difference, *p<0.05; **p<0.01. For the comparison of the total histological score of the group administered with 300 mg/kg Adenostemma lavenia extract 0805-F to the total histological score of the group administered with cyclosporine A, statistical analysis was performed by Mann-Whitney test. p<0.05 represents that there is a statistically significant difference, #p<0.05.

FIG. 8 shows the measurement results of mouse serum IgE concentration of each group in the experiment of atopic dermatitis model in NC/Nga mice. Data are presented as mean±standard error (n=5). Statistical analysis was performed by Student's t-test. For comparison of the group administered with Adenostemma lavenia extract F4AB to the group administered with its vehicle, p<0.05 represents that there is a statistically significant difference, *p<0.05; **p<0.01. For comparison of the group administered with upadacitinib to the group administered with its vehicle, p<0.05 represents that there is a statistically significant difference, #p<0.05; ##p<0.01.

FIG. 9 shows the photographs of the backs of mice in each group before sacrifice in the experiment of atopic dermatitis model in NC/Nga mice, on the 42^{nd} day of the experiment.

FIG. 10A shows the total dermatitis clinical severity score of the back of the mice administered with Adenostemma lavenia extract F4AB in the experiment of atopic dermatitis model in NC/Nga mice. Data are presented as mean±standard error (n=5). Statistical analysis was performed by Mann-Whitney test. Compared to the group administered with its vehicle, p<0.05 represents that there is a statistically significant difference, *p<0.05; **p<0.01.

FIG. 10B shows the total dermatitis clinical severity score of the back of mice administered with upadacitinib in the experiment of atopic dermatitis model in NC/Nga mice. Data are presented as mean±standard error (n=5). Statistical analysis was performed by Mann-Whitney test. Compared to the group administered with its vehicle, p<0.05 represents that there is a statistically significant difference, #p<0.05; ##p<0.01.

FIG. 10C shows the results of the total dermatitis clinical severity scores (scores of skin damage) on the back of the mice before sacrifice in the experiment of atopic dermatitis model in NC/Nga mice on the 42^{nd} day of the experiment. Data are presented as mean±standard error (n=5). Statistical analysis was performed by Mann-Whitney test. For comparison of the group administered with Adenostemma lavenia extract F4AB to the group administered with its vehicle, p<0.05 represents that there is a statistically significant difference, ***p<0.001. For comparison of the group administered with upadacitinib to the group administered with its vehicle, p<0.05 represents that there is a statistically significant difference, ###p<0.001.

FIG. 11 shows the hematoxylin-eosin staining results of the dorsal skin tissue sections of mice in the group without sensitization treatment, the group administered with Adenostemma lavenia extract F4AB and the group administered with the vehicle in the experiment of atopic dermatitis model in NC/Nga mice. Position marked by an arrow indicates hyperplasia and position marked by * indicates inflammation.

FIG. 12 shows the hematoxylin-eosin staining results of the dorsal skin tissue sections of the mice in the group without sensitization treatment, the group administered with upadacitinib, and the group administered with the vehicle in the experiment of atopic dermatitis model in NC/Nga mice. Position marked by * indicates inflammation.

FIG. 13 shows epidermal thicknesses of the dorsal skin of the mice in the group without sensitization treatment, the group administered with the vehicle for Adenostemma lavenia, the group administered with Adenostemma lavenia extract F4AB, the group administered with the vehicle for upadacitinib, the group administered with upadacitinib in the experiment of atopic dermatitis model in NC/Nga mice. Data are presented as mean±standard error (n=5). Statistical analysis was performed by Student's t-test. For comparison of the group administered with Adenostemma lavenia extract F4AB to the group administered with its vehicle, p<0.05 represents that there is a statistically significant difference, **p<0.01. For comparison of the group administered with upadacitinib to the group administered with its vehicle, p<0.05 represents that there is a statistically significant difference, ##p<0.01.

FIG. 14A shows the inflammation score of the histopathological section analysis for the dorsal skin of the mice in the experiment of atopic dermatitis model in NC/Nga mice. Data are presented as mean±standard error (Mean±SEM) (n=5), and statistical analysis was performed by Mann-Whitney test. For comparison of the group administered with represents that there is a statistically significant difference, **p<0.01. For comparison of the group administered with upadacitinib to the group administered with its vehicle, p<0.05 represents that there is a statistically significant difference, ##p<0.01.

FIG. 14B shows the necrosis score of the histopathological section analysis for the dorsal skin of the mice in the experiment of atopic dermatitis model in NC/Nga mice. Data are presented as mean±standard error (Mean±SEM) (n=5), and statistical analysis was performed by Mann-Whitney test. For comparison of the group administered with Adenostemma lavenia extract F4AB to the group administered with its vehicle, p<0.05 represents that there is a statistically significant difference, *p<0.05; **p<0.01. For comparison of the group administered with upadacitinib to the group administered with its vehicle, p<0.05 represents that there is a statistically significant difference, #p<0.05; ##p<0.01.

FIG. 14C shows the hyperplasia score of the histopathological section analysis for the dorsal skin of the mice in the experiment of atopic dermatitis model in NC/Nga mice. Data are presented as mean #standard error (Mean±SEM) (n=5), and statistical analysis was performed by Mann-Whitney test. For comparison of the group administered with Adenostemma lavenia extract F4AB to the group administered with its vehicle, p<0.05 represents that there is a statistically significant difference, *p<0.05; **p<0.01. For comparison of the group administered with upadacitinib to the group administered with its vehicle, p<0.05 represents that there is a statistically significant difference, ##p<0.01.

FIG. 14D shows the hyperkeratosis score of the histopathological section analysis for the dorsal skin of the mice in the experiment of atopic dermatitis model in NC/Nga mice. Data are presented as mean±standard error (Mean±SEM) (n=5), and statistical analysis was performed by Mann-Whitney test. For comparison of the group administered with represents that there is a statistically significant difference, *p<0.05; **p<0.01. For comparison of the group administered with upadacitinib to the group administered with its vehicle, p<0.05 represents that there is a statistically significant difference, #p<0.05; ##p<0.01.

FIG. 14E shows the skin total histopathological severity score of the histopathological section analysis for the dorsal skin of the mice in the experiment of atopic dermatitis model in NC/Nga mice. Data are presented as mean±standard error (Mean±SEM) (n=5), and statistical analysis was performed by Mann-Whitney test. For comparison of the group administered with Adenostemma lavenia extract F4AB to the group administered with its vehicle, p<0.05 represents that there is a statistically significant difference, **p<0.01. For comparison of the group administered with upadacitinib to the group administered with its vehicle, p<0.05 represents that there is a statistically significant difference, ##p<0.01.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The present disclosure may provide an Adenostemma lavenia extract. The Adenostemma lavenia extract provided by the present disclosure has an effect of inhibiting cell secretion of IL-4 and is at least able to treat and/or alleviate a symptom of dermatitis.

Example of the dermatitis mentioned above may comprise, but is not limited to, atopic dermatitis, contact dermatitis, or any combination thereof. In one embodiment, the dermatitis mentioned above may be atopic dermatitis. In another embodiment, the dermatitis mentioned above may be contact dermatitis.

Moreover, the symptom of dermatitis mentioned above may comprise erythema, hemorrhage, scarring, dryness, edema, excoriation, erosion, inflammation, necrosis, hyperplasia, hyperkeratosis, epidermal thickening, itching, etc., or any combination thereof, but it is not limited thereto.

The Adenostemma lavenia extract mentioned above may be derived from any organ or part of Adenostemma lavenia, and has no particular limitation. For example, the Adenostemma lavenia extract mentioned above may be derived from a whole plant, a root, a stem, a leaf, a flower, a seed of Adenostemma lavenia, or any combination thereof.

Furthermore, the Adenostemma lavenia extract mentioned above may be obtained by using any extraction solvent and/or any extraction method, as long as the obtained Adenostemma lavenia extract can treat and/or alleviate a symptom of dermatitis, and does not cause harm to human or animal bodies. For example, the Adenostemma lavenia extract mentioned above may be obtained by performing a single extraction using an extraction solvent, or may also be obtained by performing multiple extractions sequentially using the same or different extraction solvents. The extraction solvent mentioned above may include, but is not limited to, water, alcohol, ester, alkane, etc., or any combination thereof in any proportion. Example of the alcohol mentioned above may comprise, but is not limited to, methanol or ethanol. Example of the ester mentioned above may comprise ethyl acetate, but is not limited thereto. Example of the alkane mentioned above may comprise, but is not limited to, n-hexane, cyclohexane or heptane.

In one embodiment, the Adenostemma lavenia extract mentioned above may be an ethanol extract of Adenostemma lavenia.

An extraction method for the foregoing ethanol extract of Adenostemma lavenia has no particular limitation, as long as the obtained ethanol extract of Adenostemma lavenia can treat and/or alleviate a symptom of dermatitis, and does not cause harm to human or animal bodies.

In one embodiment, the foregoing extraction method for the ethanol extract of Adenostemma lavenia may comprise the following steps, but it is not limited thereto.

First, a plant material of Adenostemma lavenia is provided. A source of the foregoing plant material of Adenostemma lavenia may comprise a whole plant, a root, a stem, a leaf, a flower, a seed of Adenostemma lavenia, or any combination thereof, but it is not limited thereto. In one embodiment, a source of the foregoing plant material of Adenostemma lavenia may be a leaf of Adenostemma lavenia. The source of the foregoing plant material of Adenostemma lavenia may be directly used as the foregoing plant material of Adenostemma lavenia. Alternatively, the source of the foregoing plant material of Adenostemma lavenia may also be subjected to a pre-treatment and then used as the foregoing plant material of Adenostemma lavenia. The pre-treatment mentioned above may comprise, but is not limited to, drying, slicing, pulverizing, packing in a filter bag, etc. or any combination thereof in any order.

Next, a hot extraction procedure may be performed on the plant material of Adenostemma lavenia, and then a solid-liquid separation procedure may be performed to obtain an extract solution and produce a raffinate of the plant material of Adenostemma lavenia. In the hot extraction procedure, a first ethanol solvent may be used as an extraction solvent. Moreover, the extract solution may be used as the ethanol extract of Adenostemma lavenia mentioned above.

In the hot extraction procedure mentioned above, the ethanol concentration of the aforementioned first ethanol solvent has no particular limitation, and can be adjusted as needed. For example, the ethanol concentration of the aforementioned first ethanol solvent may be adjusted according to the external environment at the time of performing the extraction procedure (such as temperature, humidity, pressure of the external environment), the type of the source of the plant material of Adenostemma lavenia (such as whole plant, root, stem, leaf, flower, seed), the condition of the plant material of Adenostemma lavenia (such as water content, weight), the weight ratio of the first ethanol solvent to the plant material of Adenostemma lavenia to be used, the extraction temperature to be used and/or the extraction time to be performed, etc., but it is not limited thereto. In one embodiment, the ethanol concentration of the aforementioned first ethanol solvent may be about 30-100 vol %, such as about 40-95 vol %, about 45-90 vol %, about 50-85 vol %, about 60-80 vol %, about 70-75 vol %, about 30 vol %, about 40 vol %, about 45 vol %, about 50 vol %, about 55 vol %, about 60 vol %, about 65 vol %, about 70 vol %, about 75 vol %, about 80 vol %, about 85 vol %, about 90 vol %, about 95 vol %, but it is not limited thereto. In one embodiment, the ethanol concentration of the aforementioned first ethanol solvent may be about 95%. Furthermore, in one embodiment, the aforementioned first ethanol solvent may be an ethanol aqueous solution, but it is also not limited thereto.

In addition, in the hot extraction procedure mentioned above, the weight ratio of the foregoing first ethanol solvent to the foregoing plant material of Adenostemma lavenia also has no particular limitation, and can be adjusted as needed. For example, the weight ratio of the foregoing first ethanol solvent to the foregoing plant material of Adenostemma lavenia may be adjusted according to the external environment at the time of performing the extraction procedure (such as temperature, humidity, pressure of the external environment), the type of the source of the plant material of Adenostemma lavenia (such as whole plant, root, stem, leaf, flower, seed), the condition of the plant material of Adenostemma lavenia (such as water content, weight), the ethanol concentration of the first ethanol solvent to be used, the extraction temperature to be used and/or the extraction time to be performed, etc., but it is not limited thereto. In one embodiment, the weight ratio of the foregoing first ethanol solvent to the foregoing plant material of Adenostemma lavenia may be about 5-15:1, such as about 6-12:1, about 7-11:1, about 5:1, about 6:1, about 7:1, about 8:1, about 9:1, about 10:1, about 11:1, about 12:1, about 13:1, about 14:1, about 15:1, but it is not limited thereto. In one specific embodiment, the weight ratio of the foregoing first ethanol solvent to the foregoing plant material of Adenostemma lavenia may be about 8:1.

Moreover, the extraction temperature used in the hot extraction procedure mentioned above also has no particular limitation, as long as it is higher than the room temperature at the time of operation, and can be adjusted as needed. For example, the extraction temperature used in the hot extraction procedure mentioned above may be adjusted according to the external environment at the time of performing the extraction procedure (such as temperature, humidity, pressure of the external environment), the type of the source of the plant material of Adenostemma lavenia (such as whole plant, root, stem, leaf, flower, seed), the condition of the plant material of Adenostemma lavenia (such as water content, weight), the ethanol concentration of the first ethanol solvent to be used, the weight ratio of the first ethanol solvent to the plant material of Adenostemma lavenia to be used and/or the extraction time to be performed, etc., but it is not limited thereto. In one embodiment, the temperature used in the hot extraction procedure mentioned above may be about 40-80° C., such as about 45-75° C., about 50-70° C., about 55-65° C., about 40° C., about 45° C., about 50° C., about 55° C., about 60° C., about 65° C., about 70° C., about 75° C., about 80° C., but it is not limited thereto. In one specific embodiment, the temperature used in the hot extraction procedure mentioned above may be about 60° C.

Similarly, the time for performing the hot extraction procedure mentioned above also has no particular limitation, and can be adjusted as needed. For example, the time for performing the hot extraction procedure mentioned above may be adjusted according to the external environment at the time of performing the extraction procedure (such as temperature, humidity, pressure of the external environment), the type of the source of the plant material of Adenostemma lavenia (such as whole plant, root, stem, leaf, flower, seed), the condition of the plant material of Adenostemma lavenia (such as water content, weight), the ethanol concentration of the first ethanol solvent to be used, the weight ratio of the first ethanol solvent to the plant material of Adenostemma lavenia to be used and/or the extraction temperature to be used, etc., but it is not limited thereto. In one embodiment, the time for performing the hot extraction procedure mentioned above may be about 1-15 hours, such as about 2-12 hours, about 3-10 hours, about 4-8 hours, about 5-6 hours, about 1 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, but it is not limited thereto. In one specific embodiment, the time for performing the hot extraction procedure mentioned above may be about 8 hours.

Furthermore, the foregoing solid-liquid separation procedure also has no particular limitation, as long as the foregoing extract solution and the foregoing raffinate of the plant material of Adenostemma lavenia can be respectively obtained after the hot extraction procedure mentioned above. Example of the foregoing solid-liquid separation procedure may comprise, but is not limited to, centrifugation, filtration, or any combination thereof. In one embodiment, the foregoing solid-liquid separation procedure may be a filtration procedure.

In another embodiment, the extraction method for the ethanol extract of Adenostemma lavenia mentioned above, after the foregoing step of performing a hot extraction procedure on the plant material of Adenostemma lavenia and then performing a solid-liquid separation procedure, may further comprise performing a drying procedure on the obtained extract solution mentioned above to form a dry extract, but it is also not limited thereto. In addition, the dry extract obtained herein may be used as the ethanol extract of Adenostemma lavenia mentioned above.

The foregoing drying procedure has no particular limitation, as long as it can allow the above-mentioned extract solution to be dried into a dry extract, and has no negative influence on the extract solution in the effect of treating and/or alleviating a symptom of dermatitis. For example, the foregoing drying procedure may comprise oven drying, rotary drying, concentrating and drying under reduced pressure, but it is not limited thereto. In one embodiment, the aforementioned drying procedure may be a procedure of concentrating and drying under reduced pressure.

In another embodiment, the extraction method for the ethanol extract of Adenostemma lavenia mentioned above, after the foregoing step of performing a hot extraction procedure on the plant material of Adenostemma lavenia and then performing a solid-liquid separation procedure obtain an extract solution and produce a raffinate of the plant material of Adenostemma lavenia, may further comprise, but is not limited to, using the raffinate of the plant material of Adenostemma lavenia mentioned above as the plant material of Adenostemma lavenia, and repeating the foregoing step of performing a hot extraction procedure on the plant material of Adenostemma lavenia and then performing a solid-liquid separation procedure to obtain an extract solution and produce a raffinate of the plant material of Adenostemma lavenia at least one time, and combining all the obtained extract solutions to obtain a combined extract solution.

The related description for the hot extraction and the solid-liquid separation procedure in the repeated step of performing a hot extraction procedure on the plant material of Adenostemma lavenia and then performing a solid-liquid separation procedure to obtain an extract solution and produce a raffinate of the plant material of Adenostemma lavenia (such as the manner, operation parameters/conditions to be used) can refer to the description related to the hot extraction procedure and solid-liquid separation procedure mentioned above, and thus details will not be repeated here.

In addition, the manner, operation parameters/conditions (such as the ethanol concentration of the first ethanol solvent, the weight ratio of the first ethanol solvent to the plant material of Adenostemma lavenia, the temperature used in the hot extraction procedure, the time for performing the hot extraction procedure) to be used in the hot extraction and/or the solid-liquid separation procedure in the step of performing a hot extraction procedure on the plant material of Adenostemma lavenia and then performing a solid-liquid separation procedure to obtain an extract solution and produce a raffinate of the plant material of Adenostemma lavenia when repeated and the manner, operation parameters/conditions to be used in the hot extraction and/or the solid-liquid separation procedure in the step of performing a hot extraction procedure on the plant material of Adenostemma lavenia and then performing a solid-liquid separation procedure to obtain an extract solution and produce a raffinate of the plant material of Adenostemma lavenia when performed for the first time, depending on the need, or depending on the conditions at the time of operation, may be the same or different, and have no particular limitation. In one embodiment, the manner, operation parameters/conditions to be used in the hot extraction and/or the solid-liquid separation procedure in the step of performing a hot extraction procedure on the plant material of Adenostemma lavenia and then performing a solid-liquid separation procedure to obtain an extract solution and produce a raffinate of the plant material of Adenostemma lavenia when repeated and the manner, operation parameters/conditions to be used in the hot extraction and/or the solid-liquid separation procedure in the step of performing a hot extraction procedure on the plant material of Adenostemma lavenia and then performing a solid-liquid separation procedure to obtain an extract solution and produce a raffinate of the plant material of Adenostemma lavenia when performed for the first time are the same.

Furthermore, the manners, operation parameters/conditions (such as the ethanol concentration of the first ethanol solvent, the weight ratio of the first ethanol solvent to the plant material of Adenostemma lavenia, the temperature used in the hot extraction procedure, the time for performing the hot extraction procedure) to be used in the respective hot extraction and/or the respective solid-liquid separation procedure in the respective repeated steps of performing a hot extraction procedure on the plant material of Adenostemma lavenia and then performing a solid-liquid separation procedure to obtain an extract solution and produce a raffinate of the plant material of Adenostemma lavenia, depending on the need, or depending on the conditions at the time of operation, also may be the same or different, and have no particular limitation. In one embodiment, the manners, operation parameters/conditions (such as the ethanol concentration of the first ethanol solvent, the weight ratio of the first ethanol solvent to the plant material of Adenostemma lavenia, the temperature used in the hot extraction procedure, the time for performing the hot extraction procedure) to be used in the respective hot extraction and/or the respective solid-liquid separation procedure in the respective repeated steps of performing a hot extraction procedure on the plant material of Adenostemma lavenia and then performing a solid-liquid separation procedure to obtain an extract solution and produce a raffinate of the plant material of Adenostemma lavenia are the same.

In further another embodiment, the extraction method for the ethanol extract of Adenostemma lavenia mentioned above, after the foregoing step of combining all the obtained extract solutions to obtain a combined extract solution, may further comprise, performing a drying procedure on the combined extract solution to obtain a dry extract, but it is also not limited thereto. The drying procedure mentioned herein has no particular limitation, as long as it can allow the above-mentioned combined extract solution to be dried into a dry extract, and has no negative influence on the combined extract solution in the effect of treating and/or alleviating a symptom of dermatitis. For example, the drying procedure mentioned herein may comprise oven drying, rotary drying, concentrating and drying under reduced pressure, but it is not limited thereto. In one embodiment, the drying procedure mentioned herein may be a procedure of concentrating and drying under reduced pressure. Moreover, the dry extract obtained herein may be used as the ethanol extract of Adenostemma lavenia mentioned above.

In addition, in one specific embodiment, the extraction method for the ethanol extract of Adenostemma lavenia mentioned above, after the foregoing step of performing a drying procedure on the combined extract solution to obtain a dry extract, may further comprise, the following steps, but it is also not limited thereto.

First, a heating procedure is performed on the foregoing dry extract in a second ethanol solvent until the foregoing dry extract is dissolved to form a mixture.

In the heating procedure mentioned above, the ethanol concentration of the aforementioned second ethanol solvent also has no particular limitation, and can be adjusted as needed. For example, the ethanol concentration of the aforementioned second ethanol solvent may be adjusted according to the external environment at the time of performing the heating procedure (such as temperature, humidity, pressure of the external environment), the condition of the dry extract (such as water content, weight), the weight ratio of the second ethanol solvent to the dry extract to be used, the heating temperature to be used and/or the heating time to be performed, etc., but it is not limited thereto. In one embodiment, the ethanol concentration of the aforementioned second ethanol solvent may be about 30-100 vol %, such as about 40-95 vol %, about 45-90 vol %, about 50-85 vol %, about 60-80 vol %, about 70-75 vol %, about 30 vol %, about 40 vol %, about 45 vol %, about 50 vol %, about 55 vol %, about 60 vol %, about 65 vol %, about 70 vol %, about 75 vol %, about 80 vol %, about 85 vol %, about 90 vol %, about 95 vol %, but it is not limited thereto. In one embodiment, the ethanol concentration of the aforementioned second ethanol solvent may be about 95%. Moreover, in one embodiment, the aforementioned second ethanol solvent may be an ethanol aqueous solution, but it is also not limited thereto.

Furthermore, in the heating procedure mentioned above, the weight ratio of the aforementioned second ethanol solvent to the aforementioned dry extract also has no particular limitation, and can be adjusted as needed. For example, the weight ratio of the aforementioned first ethanol solvent to the aforementioned dry extract may be adjusted according to the external environment at the time of performing the heating procedure (such as temperature, humidity, pressure of the external environment), the condition of the dry extract (such as water content, weight), the ethanol concentration of the second ethanol solvent to be used, the heating temperature to be used and/or the heating time to be performed, etc., but it is not limited thereto. In one embodiment, the weight ratio of the aforementioned second ethanol solvent to the aforementioned dry extract may be about 5-15:1, such as about 6-12:1, about 7-11:1, about 5:1, about 6:1, about 7:1, about 8:1, about 9:1, about 10:1, about 11:1, about 12:1, about 13:1, about 14:1, about 15:1, but it is not limited thereto. In one specific embodiment, the weight ratio of the aforementioned second ethanol solvent to the aforementioned dry extract may be about 8:1.

Furthermore, the temperature used in the heating procedure mentioned above also has no particular limitation, as long as it is higher than the room temperature at the time of operation, and can be adjusted as needed. For example, the temperature used in the heating procedure mentioned above may be adjusted according to the external environment at the time of performing the heating procedure (such as temperature, humidity, pressure of the external environment), the condition of the dry extract (such as water content, weight), the ethanol concentration of the second ethanol solvent to be used, the weight ratio of the second ethanol solvent to the dry extract to be used and/or the heating time to be performed, etc., but it is not limited thereto. In one embodiment, the temperature used in the heating procedure mentioned above may be about 30-70° C., such as about 35-65° C., about 40-60° C., about 45-55° C., about 30° C., about 35° C., about 40° C., about 45° C., about 50° C., about 55° C., about 60° C., about 65° C., about 70° C., but it is not limited thereto. In one specific embodiment, the temperature used in the heating procedure mentioned above may be about 50° C.

Next, after the mixture mentioned above is formed, column chromatography is performed on the mixture mentioned above, and at least one fraction having an effect of inhibiting cell secretion of IL-4 is collected or collected and combined as an active fraction. Moreover, the active fraction obtained herein may be used as the ethanol extract of Adenostemma lavenia mentioned above.

In the column chromatography mentioned above, a solvent which can be used as a mobile phase has no particular limitation, as long as it can obtain at least one fraction having an effect of inhibiting cell secretion of IL-4 and having no negative impact on the human bodies or animal bodies. A solvent which can be used as a mobile phase may comprise, but is not limited to, water, alcohol, ester, alkane, etc., or any combination thereof in any proportion. Example of the alcohol mentioned above may comprise, but is not limited to, methanol or ethanol. Example of the ester mentioned above may comprise ethyl acetate, but is not limited thereto. Example of the alkane mentioned above may comprise, but is not limited to, n-hexane, cyclohexane or heptane. In one embodiment, in the column chromatography mentioned above, a methanol solvent can be used as a mobile phase. In another embodiment, in the column chromatography mentioned above, a mixed solvent of n-hexane/ethyl acetate may be used as a mobile phase. The volume ratio of n-hexane to ethyl acetate of the mixed solvent of n-hexane/ethyl acetate mentioned above also has no particular limitation, as long as it can obtain at least one fraction having an effect of inhibiting cell secretion of IL-4 and having no negative impact on the human bodies or animal bodies. For example, the volume ratio of n-hexane to ethyl acetate of the mixed solvent of n-hexane/ethyl acetate mentioned above may be about 0.1-1:1, such as 0.2-0.8:1, 0.3-0.7:1, 0.4-0.5:1, 0.1:1, 0.2:1, 0.25:1, 0.3:1, 0.4:1, 0.5:1, 0.6:1, 0.7:1, 0.8:1, 0.9:1, 1:1, but it is not limited thereto.

The type of the column chromatography mentioned above has no particular limitation. In one embodiment, the column chromatography mentioned above may be silica gel column chromatography. In another embodiment, the column chromatography mentioned above may be high performance liquid chromatography (HPLC), but it is not limited thereto.

In further another specific embodiment, the extraction method for the ethanol extract of Adenostemma lavenia mentioned above, after collecting, or collecting and combining at least one fraction having an effect of inhibiting cell secretion of IL-4 as an active fraction, may further comprise, performing a drying procedure on the active fraction to obtain a dry active fraction, but it is also not limited thereto. Furthermore, the dry active fraction obtained herein may be used as the ethanol extract of Adenostemma lavenia mentioned above.

In addition, in another embodiment of the present disclosure, the Adenostemma lavenia extract mentioned above may be a water extract of Adenostemma lavenia.

An extraction method for the water extract of Adenostemma lavenia mentioned above has no particular limitation, as long as the obtained water extract of Adenostemma lavenia can treat and/or alleviate a symptom of dermatitis, and does not cause harm to human or animal bodies.

In one embodiment, the foregoing extraction method for the water extract of Adenostemma lavenia may comprise the following steps, but it is not limited thereto.

First, a plant material of Adenostemma lavenia is provided. A source of the foregoing plant material of Adenostemma lavenia may comprise a whole plant, a root, a stem, a leaf, a flower, a seed of Adenostemma lavenia, or any combination thereof, but it is not limited thereto. In one embodiment, a source of the foregoing plant material of Adenostemma lavenia may be a leaf of Adenostemma lavenia. The source of the foregoing plant material of Adenostemma lavenia may be directly used as the foregoing plant material of Adenostemma lavenia. Alternatively, the source of the foregoing plant material of Adenostemma lavenia may also be subjected to a pre-treatment and then used as the foregoing plant material of Adenostemma lavenia. The pre-treatment mentioned above may comprise, but is not limited to, drying, slicing, pulverizing, packing in a filter bag, etc. or any combination thereof in any order.

Next, a pressurized extraction procedure may be performed on the plant material of Adenostemma lavenia, and then a solid-liquid separation procedure may be performed to obtain an extract solution and produce a raffinate of the plant material of Adenostemma lavenia. In the pressurized extraction procedure, water may be used as an extraction solvent. Moreover, the extract solution obtained herein may be used as the water extract of Adenostemma lavenia mentioned above.

In the pressurized extraction procedure mentioned above, the weight ratio of the aforementioned water to the aforementioned plant material of Adenostemma lavenia also has no particular limitation, and can be adjusted as needed. For example, the weight ratio of the aforementioned water to the aforementioned plant material of Adenostemma lavenia may be adjusted according to the external environment at the time of performing the extraction procedure (such as temperature, humidity, pressure of the external environment), the type of the source of the plant material of Adenostemma lavenia (such as whole plant, root, stem, leaf, flower, seed), the condition of the plant material of Adenostemma lavenia (such as water content, weight), the extraction pressure to be used, the extraction temperature to be used and/or the extraction time to be performed, etc., but it is not limited thereto. In one embodiment, the weight ratio of the aforementioned water to the aforementioned plant material of Adenostemma lavenia may be about 10-50:1, such as about 15-45:1, about 20-40:1, about 25-35:1, about 15:1, about 20:1, about 25:1, about 30:1, about 35:1, about 40:1, about 45:1, about 50:1, but it is not limited thereto. In one specific embodiment, the weight ratio of the aforementioned water to the aforementioned plant material of Adenostemma lavenia may be about 30:1.

The pressure used in the pressurized extraction procedure mentioned above has no particular limitation, and can be adjusted as needed. For example, the pressure used in the pressurized extraction procedure mentioned above may be adjusted according to the external environment at the time of performing the pressurized extraction procedure (such as temperature, humidity, pressure of the external environment), the type of the source of the plant material of Adenostemma lavenia (such as whole plant, root, stem, leaf, flower, seed), the condition of the plant material of Adenostemma lavenia (such as water content, weight), the weight ratio of water to the plant material of Adenostemma lavenia to be used, the extraction temperature to be used and/or the extraction time to be performed, etc., but it is not limited thereto. In one embodiment, the pressure used in the pressurized extraction procedure mentioned above may be about 101.4-140.0 kPa, such as about 101.5-135.0 kPa, about 101.6-130.0 kPa, about 101.8-125.0 kPa, about 102.0-120.0 kPa, about 102.2-115.0 kPa, about 102.4-110.0 kPa, about 102.6-105.0 kPa, about 102.8-104.0 kPa, about 101.5 kPa, about 101.6 kPa, about 101.7 kPa, about 101.8 kPa, about 101.9 kPa, about 102.0 kPa, about 102.1 kPa, about 102.2 kPa, about 102.25 kPa, about 102.3 kPa, about 102.4 kPa, about 102.5 kPa, about 102.6 kPa, about 102.7 kPa, about 102.8 kPa, about 102.9 kPa, about 102.92 kPa, about 102.94 kPa, about 102.96 kPa, about 102.97 kPa, about 102.98 kPa, about 103.0 kPa, about 105.0 kPa, about 107.0 kPa, about 108.0 kPa, about 110.0 kPa, about 112.0 kPa, about 115.0 kPa, about 120.0 kPa, about 125.0 kPa, about 130.0 kPa, about 135.0 kPa, about 140.0 kPa, but it is not limited thereto. In one specific embodiment, the pressure used in the pressurized extraction procedure mentioned above may be about 102.97 kPa.

The extraction temperature used in the pressurized extraction procedure mentioned above has no particular limitation, and can be adjusted as needed. For example, the extraction temperature used in the pressurized extraction procedure mentioned above may be adjusted according to the external environment at the time of performing the pressurized extraction procedure (such as temperature, humidity, pressure of the external environment), the type of the source of the plant material of Adenostemma lavenia (such as whole plant, root, stem, leaf, flower, seed), the condition of the plant material of Adenostemma lavenia (such as water content, weight), the weight ratio of water to the plant material of Adenostemma lavenia to be used, the extraction pressure to be used and/or the extraction time to be performed, etc., but it is not limited thereto. In one embodiment, the extraction temperature used in the pressurized extraction procedure mentioned above may be about 100-140° C., such as about 102-135° C., about 105-130° C., about 108-125° C., about 110-120° C., about 100° C., about 101° C., about 103° C., about 105° C., about 110° C., about 112° C., about 115° C., about 120° C., etc., but it is not limited thereto. In one specific embodiment, the extraction temperature used in the pressurized extraction procedure mentioned above may be about 120° C.

The time for performing the pressurized extraction procedure mentioned above also has no particular limitation, and can be adjusted as needed. For example, the time for performing the pressurized extraction procedure mentioned above may be adjusted according to the external environment at the time of performing the pressurized extraction procedure (such as temperature, humidity, pressure of the external environment), the type of the source of the plant material of Adenostemma lavenia (such as whole plant, root, stem, leaf, flower, seed), the condition of the plant material of Adenostemma lavenia (such as water content, weight), the weight ratio of water to the plant material of Adenostemma lavenia to be used, the extraction pressure to be used and/or the extraction temperature to be used, etc., but it is not limited thereto. In one embodiment, the time for performing the pressurized extraction procedure mentioned above may be about 5-60 minutes, about 10-55 minutes, about 15-50 minutes, about 20-45 minutes, about 25-40 minutes, about 30-35 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, etc., but it is not limited thereto. In one specific embodiment, the time for performing the pressurized extraction procedure mentioned above may be about 20 minutes.

In another embodiment, the extraction method for the water extract of Adenostemma lavenia mentioned above, after the foregoing step of performing a pressurized extraction procedure on the plant material of Adenostemma lavenia and then performing a solid-liquid separation procedure, may further comprise performing a drying procedure on the obtained extract solution mentioned above to form a dry extract. In addition, the dry extract obtained herein may be used as the water extract of Adenostemma lavenia mentioned above.

Similarly, the drying procedure mentioned herein has no particular limitation, as long as it can allow the above-mentioned extract solution to be dried into a dry extract, and has no negative impact on the extract solution in the effect of treating and/or alleviating a symptom of dermatitis. For example, the drying procedure mentioned herein may comprise oven drying, rotary drying, concentering and drying under reduced pressure, but it is not limited thereto.

In another embodiment, the extraction method for the water extract of Adenostemma lavenia mentioned above, after the foregoing step of performing a pressurized extraction procedure on the plant material of Adenostemma lavenia and then performing a solid-liquid separation procedure to obtain an extract solution and produce a raffinate of the plant material of Adenostemma lavenia, may further comprise, but is not limited to, using the raffinate of the plant material of Adenostemma lavenia mentioned above as the plant material of Adenostemma lavenia, and repeating the foregoing step of performing a pressurized extraction procedure on the plant material of Adenostemma lavenia and then performing a solid-liquid separation procedure to obtain an extract solution and produce a raffinate of the plant material of Adenostemma lavenia at least one time, and combining all the obtained extract solutions to obtain a combined extract solution. Moreover, the combined extract solution obtained herein may be used as the water extract of Adenostemma lavenia mentioned above. In one specific embodiment, the foregoing step of performing a pressurized extraction procedure on the plant material of Adenostemma lavenia and then performing a solid-liquid separation procedure to obtain an extract solution and produce a raffinate of the plant material of Adenostemma lavenia is repeated three times, and all extraction solutions are combined to obtain a combined extraction solution. Furthermore, the combined extract solution obtained herein may also be used as the water extract of Adenostemma lavenia mentioned above.

The related description for the pressurized extraction procedure and the solid-liquid separation procedure in the step of performing a pressurized extraction procedure on the plant material of Adenostemma lavenia and then performing a solid-liquid separation procedure to obtain an extract solution and produce a raffinate of the plant material of Adenostemma lavenia (such as the manner, operation parameters/conditions to be used) when repeated can refer to the description related to the pressurized extraction procedure and solid-liquid separation procedure mentioned above, and thus details will not be repeated here.

In addition, the manner, operation parameters/conditions (such as the weight ratio of water to the plant material of Adenostemma lavenia, the pressure used in the pressurized extraction procedure, the temperature used in the pressurized extraction procedure, the time for performing the pressurized extraction procedure) to be used in the hot extraction and/or the solid-liquid separation procedure in the step of performing a hot extraction procedure on the plant material of Adenostemma lavenia and then performing a solid-liquid separation procedure to obtain an extract solution and produce a raffinate of the plant material of Adenostemma lavenia when repeated and the manner, operation parameters/conditions to be used in the pressurized extraction procedure and/or the solid-liquid separation procedure in the step of performing a pressurized extraction procedure on the plant material of Adenostemma lavenia and then performing a solid-liquid separation procedure to obtain an extract solution and produce a raffinate of the plant material of Adenostemma lavenia when performed for the first time, depending on the need, or depending on the conditions at the time of operation, may be the same or different, and have no particular limitation. In one embodiment, the manner, operation parameters/conditions to be used in the pressurized extraction procedure and/or the solid-liquid separation procedure in the step of performing a pressurized extraction procedure on the plant material of Adenostemma lavenia and then performing a solid-liquid separation procedure to obtain an extract solution and produce a raffinate of the plant material of Adenostemma lavenia when repeated and the manner, operation parameters/conditions to be used in the pressurized extraction procedure and/or the solid-liquid separation procedure in the step of performing a hot extraction procedure on the plant material of Adenostemma lavenia and then performing a solid-liquid separation procedure to obtain an extract solution and produce a raffinate of the plant material of Adenostemma lavenia when performed for the first time are the same.

Furthermore, the manners, operation parameters/conditions (such as the weight ratio of water to the plant material of Adenostemma lavenia, the pressure used in the pressurized extraction procedure, the temperature used in the pressurized extraction procedure, the time for performing the pressurized extraction procedure) to be used in the respective pressurized extraction procedure and/or the respective solid-liquid separation procedure in the respective repeated steps of performing a pressurized extraction procedure on the plant material of Adenostemma lavenia and then performing a solid-liquid separation procedure to obtain an extract solution and produce a raffinate of the plant material of Adenostemma lavenia, depending on the need, or depending on the conditions at the time of operation, also may be the same or different, and have no particular limitation. In one embodiment, the manners, operation parameters/conditions to be used in the respective pressurized extraction procedure and/or the respective solid-liquid separation procedure in the respective repeated steps of performing a pressurized extraction procedure on the plant material of Adenostemma lavenia and then performing a solid-liquid separation procedure to obtain an extract solution and produce a raffinate of the plant material of Adenostemma lavenia are the same.

In further another embodiment, the extraction method for the water extract of Adenostemma lavenia mentioned above, after the foregoing step of combining all the obtained extract solutions to obtain a combined extract solution, may further comprise, but is not limited to, performing a drying procedure on the combined extract solution to obtain a dry extract. Moreover, the dry extract obtained herein also may be used as the water extract of Adenostemma lavenia mentioned above. The drying procedure mentioned herein has no particular limitation, as long as it can allow the above-mentioned combined extract solution to be dried into a dry extract, and has no negative influence on the combined extract solution in the effect of treating and/or alleviating a symptom of dermatitis. For example, the drying procedure mentioned herein may comprise oven drying, rotary drying, concentrating and drying under reduced pressure, but it is not limited thereto. In one embodiment, the drying procedure mentioned herein may be a procedure of concentrating and drying under reduced pressure.

Furthermore, in one specific embodiment, the extraction method for the water extract of Adenostemma lavenia mentioned above, after the foregoing step of combining all the obtained extract solutions to obtain a combined extract solution, may further comprise, performing a first column chromatography on the combined extract solution mentioned above, but it is also not limited thereto. The foregoing first column chromatography may comprise the following steps, but it is also not limited thereto.

First, the combined extract solution mentioned above is filled into a column packed with resin. The type of the resin packed in the column mentioned above has no particular limitation, as long as it can absorb an active ingredient in the combined extract solution. For example, the type of the resin packed in the column mentioned above may comprise styrene-divinylbenzene series adsorption resin (such as HP20, DIAION), etc., but it is not limited thereto.

Next, after the combined extract solution mentioned above is filled into a column packed with resin, the column is eluted with water, and the eluate that flows out from the column is discarded.

The volume ratio of water used to elute the column mentioned above and the combined extract solution filled into the column mentioned above also has no particular limitation, and can be adjusted as needed. For example, the volume ratio of water used to elute the column mentioned above and the combined extract solution filled into the column mentioned above may be adjusted according to the environment at the time of eluting the column (such as temperature, humidity, pressure of the environment), the amount of the combined extract solution filled into the column, the condition of the eluate flowed out from the column at the time of eluting the column, but it is not limited thereto. In one embodiment, the volume ratio of water used to elute the column mentioned above and the combined extract solution filled into the column mentioned above may be about 0.5-10:1, such as about 1-8:1, about 1.5-7:1, about 2-6:1, about 2.5-5:1, about 3-4:1, about 0.5:1, about 1:1, about 2:1, about 2.5:1, about 3:1, about 4:1, about 5:1, about 6:1, about 7:1, about 8:1, about 9:1, about 10:1, but it is not limited thereto. In one specific embodiment, the volume ratio of water used to elute the column mentioned above and the combined extract solution filled into the column mentioned above may be 1:1.

Then, after the column is eluted with water and the eluate that flows out from the column is discarded, the column is eluted with a third ethanol solvent, and the eluate that flows out from the column is discarded.

The volume ratio of the third ethanol solvent used to elute the column mentioned above and the combined extract solution filled into the column mentioned above also has no particular limitation, and can be adjusted as needed. For example, the volume ratio of the third ethanol solvent used to elute the column mentioned above and the combined extract solution filled into the column mentioned above may be adjusted according to the environment at the time of eluting the column (such as temperature, humidity, pressure of the environment), the amount of the combined extract solution filled into the column, the ethanol concentration of the third ethanol solvent, the foregoing condition of the eluate flowing out from the column by eluting the column with water and/or the condition at the time of the eluate flowing out of the column by eluting the column with the third ethanol solvent, but it is not limited thereto. In one embodiment, the volume ratio of the third ethanol solvent used to elute the column mentioned above and the combined extract solution filled into the column mentioned above may be about 0.5-10:1, such as about 1-8:1, about 1.5-7:1, about 2-6:1, about 2.5-5:1, about 3-4:1, about 0.5:1, about 1:1, about 2:1, about 2.5:1, about 3:1, about 4:1, about 5:1, about 6:1, about 7:1, about 8:1, about 9:1, about 10:1, but it is not limited thereto. In one specific embodiment, the volume ratio of the third ethanol solvent used to elute the column mentioned above and the combined extract solution filled into the column mentioned above may be 1:1.

The ethanol concentration of the third ethanol solvent used to elute the column also has no particular limitation, and can be adjusted as needed. For example, the ethanol concentration of the third ethanol solvent may be adjusted according to the environment at the time of eluting the column (such as temperature, humidity, pressure of the environment), the amount of the combined extract solution filled into the column, the volume ratio of the third ethanol solvent used to elute the column mentioned above and the combined extract solution filled into the column mentioned above, the foregoing condition of the eluate flowing out from the column by eluting the column with water and/or the condition at the time of the eluate flowing out of the column by eluting the column with the third ethanol solvent, but it is not limited thereto. In one embodiment, the ethanol concentration of the third ethanol solvent mentioned above may be about 30-100 vol %, such as about 40-95 vol %, about 45-90 vol %, about 50-85 vol %, about 60-80 vol %, about 70-75 vol %, about 30 vol %, about 40 vol %, about 45 vol %, about 50 vol %, about 55 vol %, about 60 vol %, about 65 vol %, about 70 vol %, about 75 vol %, about 80 vol %, about 85 vol %, about 90 vol %, about 95 vol %, but it is not limited thereto. In one embodiment, the ethanol concentration of the third ethanol solvent mentioned above may be about 50%. Moreover, in one embodiment, the third ethanol solvent mentioned above may be an ethanol aqueous solution, but it is also not limited thereto.

Next, after the column is eluted with third ethanol solvent mentioned above and the eluate that flows out from the column is discarded, the column is eluted with a fourth ethanol solvent, and the eluate that flows out from the column is collected as a preliminary purified extract solution. The preliminary purified extract solution obtained herein may be used as the water extract of Adenostemma lavenia mentioned above.

Similarly, the volume ratio of the fourth ethanol solvent used to elute the column mentioned above and the combined extract solution filled into the column mentioned above also has no particular limitation, and can be adjusted as needed. For example, the volume ratio of the fourth ethanol solvent used to elute the column mentioned above and the combined extract solution filled into the column mentioned above may be adjusted according to the environment at the time of eluting the column (such as temperature, humidity, pressure of the environment), the amount of the combined extract solution filled into the column, the ethanol concentration of the fourth ethanol solvent, the foregoing condition of the eluate flowing out from the column by eluting the column with the third ethanol solvent and/or the condition at the time of the eluate flowing out of the column by eluting the column with the fourth ethanol solvent, but it is not limited thereto. In one embodiment, the volume ratio of the fourth ethanol solvent used to elute the column mentioned above and the combined extract solution filled into the column mentioned above may be about 0.5-10:1, such as about 1-8:1, about 1.5-7:1, about 2-6:1, about 2.5-5:1, about 3-4:1, about 0.5:1, about 1:1, about 2:1, about 2.5:1, about 3:1, about 4:1, about 5:1, about 6:1, about 7:1, about 8:1, about 9:1, about 10:1, but it is not limited thereto. In one specific embodiment, the volume ratio of the fourth ethanol solvent used to elute the column mentioned above and the combined extract solution filled into the column mentioned above may be 1:1.

The ethanol concentration of the fourth ethanol solvent used to elute the column also has no particular limitation, and can be adjusted as needed. For example, the ethanol concentration of the fourth ethanol solvent may be adjusted according to the environment at the time of eluting the column (such as temperature, humidity, pressure of the environment), the amount of the combined extract solution filled into the column, the volume ratio of the fourth ethanol solvent used to elute the column mentioned above and the combined extract solution filled into the column mentioned above, the foregoing condition of the eluate flowing out from the column by eluting the column with the third ethanol solvent and/or the condition at the time of the eluate flowing out of the column by eluting the column with the fourth ethanol solvent, but it is not limited thereto. In one embodiment, the ethanol concentration of the fourth ethanol solvent mentioned above may be about 30-100 vol %, such as about 40-95 vol %, about 45-90 vol %, about 50-85 vol %, about 60-80 vol %, about 70-75 vol %, about 30 vol %, about 40 vol %, about 45 vol %, about 50 vol %, about 55 vol %, about 60 vol %, about 65 vol %, about 70 vol %, about 75 vol %, about 80 vol %, about 85 vol %, about 90 vol %, about 95 vol %, but it is not limited thereto. In one embodiment, the ethanol concentration of the fourth ethanol solvent mentioned above may be about 75%. Moreover, in one embodiment, the fourth ethanol solvent mentioned above may be an ethanol aqueous solution, but it is also not limited thereto.

In further another embodiment, the extraction method for the water extract of Adenostemma lavenia mentioned above, after eluting the column with a fourth ethanol solvent, and collecting the eluate that flows out from the column as a preliminary purified extract solution, may further comprise, performing a second column chromatography on the preliminary purified extract solution mentioned above, and collecting, or collecting and combining at least one fraction having an effect of inhibiting cell secretion of IL-4 as an active fraction, but it is also not limited thereto. Furthermore, the active fraction obtained herein may be used as the water extract of Adenostemma lavenia mentioned above.

In the second column chromatography mentioned above, a solvent which can be used as a mobile phase has no particular limitation, as long as it can obtain at least one fraction having an effect of inhibiting cell secretion of IL-4 and having no negative impact on the human bodies or animal bodies. A solvent which can be used as a mobile phase may comprise, but is not limited to, water, alcohol, ester, alkane, etc., or any combination thereof in any proportion. Example of the alcohol mentioned above may comprise, but is not limited to, methanol or ethanol. Example of the ester mentioned above may comprise ethyl acetate, but is not limited thereto. Example of the alkane mentioned above may comprise, but is not limited to, n-hexane, cyclohexane or heptane. In one embodiment, in the second column chromatography mentioned above, a methanol solvent can be used as a mobile phase. In another embodiment, in the second column chromatography mentioned above, a mixed solvent of n-hexane/ethyl acetate may be used as a mobile phase. The volume ratio of n-hexane to ethyl acetate of the mixed solvent of n-hexane/ethyl acetate mentioned above also has no particular limitation, as long as it can obtain at least one fraction having an effect of inhibiting cell secretion of IL-4 and having no negative impact on the human bodies or animal bodies. For example, the volume ratio of n-hexane to ethyl acetate of the mixed solvent of n-hexane/ethyl acetate mentioned above may be about 0.1-1:1, such as 0.2-0.8:1, 0.3-0.7:1, 0.4-0.5:1, 0.1:1, 0.2:1, 0.25:1, 0.3:1, 0.4:1, 0.5:1, 0.6:1, 0.7:1, 0.8:1, 0.9:1, 1:1, but it is not limited thereto. In one specific embodiment, the volume ratio of n-hexane to ethyl acetate of the mixed solvent of n-hexane/ethyl acetate mentioned above may be about 1:1.

The type of the second column chromatography mentioned above has no particular limitation. In one embodiment, the second column chromatography mentioned above may be silica gel column chromatography. In another embodiment, the second column chromatography mentioned above may be high performance liquid chromatography (HPLC).

In further another specific embodiment, the extraction method for the water extract of Adenostemma lavenia mentioned above, after performing a second column chromatography on the preliminary purified extract solution and collecting, or collecting and combining at least one fraction having an effect of inhibiting cell secretion of IL-4 as an active fraction, may further comprise, concentrating the active fraction mentioned above to obtain a concentrated active fraction, but it is also not limited thereto. Moreover, the concentrated active fraction obtained herein may be also used as the water extract of Adenostemma lavenia mentioned above.

In addition, in this specific embodiment, the extraction method for the water extract of Adenostemma lavenia mentioned above, after concentrating the active fraction mentioned above to obtain a concentrated active fraction, may further comprise, the following steps, but it is also not limited thereto.

First, a heating and dissolving procedure is performed on the concentrated active fraction mentioned above with a fifth ethanol solvent to form an active solution.

The weight ratio of the fifth ethanol solvent to the aforementioned concentrated active fraction also has no particular limitation, and can be adjusted as needed. For example, the weight ratio of the fifth ethanol solvent to the aforementioned concentrated active fraction may be adjusted according to the external environment at the time of performing the heating and dissolving procedure (such as temperature, humidity, pressure of the external environment) and/or the condition of the concentrated active fraction mentioned above (such as water content, weight), etc., but it is not limited thereto. In one embodiment, the weight ratio of the fifth ethanol solvent to the aforementioned concentrated active fraction may be about 1-10:1, such as about 1.5-8:1, about 2-5:1, about 2.5-4.5:1, about 1:1, about 1.5:1, about 2:1, about 2.5:1, about 3:1, about 4:1, about 5:1, about 6:1, about 7:1, about 8:1, about 9:1, about 1:10, but it is not limited thereto. In one specific embodiment, the weight ratio of the fifth ethanol solvent to the aforementioned active solution may be about 4:1.

The temperature used for the heating and dissolving procedure mentioned above has no particular limitation, as long as it is higher than the room temperature and can allow the concentrated active fraction mentioned above to be dissolved, and can be adjusted as needed. For example, the temperature used for the heating and dissolving procedure mentioned above may be adjusted according to the external environment at the time of performing the heating and dissolving procedure (such as temperature, humidity, pressure of the external environment), the condition of the concentrated active fraction (such as water content, weight), the weight ratio of the fifth ethanol solvent to the aforementioned concentrated active fraction and/or the heating time to be performed, etc., but it is not limited thereto. In one embodiment, the temperature used for the heating and dissolving procedure may be about 40-100° C., such as about 45-95° C., about 50-90° C., about 55-85° C., about 60-80° C., about 65-75° C., about 40° C., about 45° C., about 50° C., about 55° C., about 60° C., about 65° C., about 70° C., about 75° C., about 80° C., about 85° C., about 90° C., about 95° C., about 100° C., but it is not limited thereto. In one specific embodiment, the temperature used for the heating and dissolving procedure may be about 60° C.

The time for performing the heating and dissolving procedure mentioned above has no particular limitation, as long as it can allow the concentrated active fraction mentioned above to be dissolved, and can be adjusted as needed. For example, the time for performing the heating and dissolving procedure mentioned above may be adjusted according to the external environment at the time of performing the heating and dissolving procedure (such as temperature, humidity, pressure of the external environment), the condition of the concentrated active fraction (such as water content, weight), the weight ratio of the fifth ethanol solvent to the aforementioned concentrated active fraction and/or the heating temperature to be used, etc., but it is not limited thereto. In one embodiment, time for performing the heating and dissolving procedure mentioned above may be about 5-60 minutes, such as about 10-55 minutes, about 15-50 minutes, about 20-45 minutes, about 25-40 minutes, about 30-35 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, but it is not limited thereto. In one specific embodiment, time for performing the heating and dissolving procedure mentioned above may be 60 minutes

Next, after a heating and dissolving procedure is performed on the concentrated active fraction mentioned above with a fifth ethanol solvent to form an active solution, the active solution is stood to make crystals form therein.

The time for standing the active solution mentioned above also has no particular limitation, as long as it can make the active solution mentioned above form crystals, and can be adjusted as needed. For example, the time for standing the active solution mentioned above may be adjusted according to the external environment at the time of performing the heating and dissolving procedure (such as temperature, humidity, pressure of the external environment), the condition of the active solution (such as weight), but it is not limited thereto. In one embodiment, the time for standing the active solution mentioned above may be about 6-48 hours, about 9-45 hours, about 12-42 hours, about 15-39 hours, about 18-36 hours, about 21-33 hours, about 24-30 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, but it is not limited thereto. In one specific embodiment, the time for standing the active solution mentioned above may be about 24 hours.

After forming the crystals mentioned above, the crystals mentioned above are collected. The crystals collected herein may be used as the water extract of Adenostemma lavenia mentioned above.

In addition, a drying procedure may be performed on the crystals collected.

The Adenostemma lavenia extract of the present disclosure obtained by any of the extraction methods mentioned above has the effect of inhibiting the cell secretion of IL-4, and can at least treat and/or alleviate a symptom of dermatitis.

Furthermore, the present disclosure also may provide a use of Adenostemma lavenia extract in the manufacture of a medicament for treating dermatitis.

The Adenostemma lavenia extract in the use of Adenostemma lavenia extract in the manufacture of a medicament for treating dermatitis of the present disclosure, has no particular limitation, as long as it has an effect of treating and/or alleviating a symptom of dermatitis. For example, any Adenostemma lavenia extract of the present disclosure mentioned above can be used as the Adenostemma lavenia extract in the use of Adenostemma lavenia extract in the manufacture of a medicament for treating dermatitis of the present disclosure.

In addition, in the use of Adenostemma lavenia extract in the manufacture of a medicament for treating dermatitis of the present disclosure, the medicament for treating dermatitis may further comprise a pharmaceutically acceptable carrier or salt, but it is not limited thereto.

The present disclosure also may provide a pharmaceutical composition for treating dermatitis.

The aforementioned pharmaceutical composition for treating dermatitis of the present disclosure may comprise, but is not limited to, an Adenostemma lavenia extract. The Adenostemma lavenia extract in the pharmaceutical composition for treating dermatitis of the present disclosure also has no particular limitation, as long as it has the effect of treating and/or alleviating a symptom of dermatitis. For example, the Adenostemma lavenia extract in the aforementioned pharmaceutical composition for treating dermatitis of the present disclosure may be any of the Adenostemma lavenia extracts of the present disclosure mentioned above.

In addition, the pharmaceutical composition for treating dermatitis of the present disclosure may further comprise a pharmaceutically acceptable carrier or salt, but it is not limited thereto.

The pharmaceutically acceptable carrier mentioned above may comprise, but is not limited to, a solvent, a dispersion medium, a coating, an antibacterial and antifungal agent, or an isotonic and absorption delaying agent, etc. which is suitable for pharmaceutical administration. The pharmaceutical composition can be formulated into dosage forms for different administration routes utilizing conventional methods.

Furthermore, the pharmaceutically acceptable salt mentioned above may include, but is not limited to, salts including inorganic cation, such as alkali metal salts such as sodium salt, potassium salt or amine salt, such as alkaline-earth metal salt such as magnesium salt or calcium salt, such as the salt containing bivalent or quadrivalent cation such as zinc salt, aluminum salt or zirconium salt. In addition, the pharmaceutically acceptable salt may also be organic salt, such as dicyclohexylamine salt, methyl-D-glucamine, and amino acid salt such as arginine, lysine, histidine, or glutamine.

In addition, the Adenostemma lavenia extract of the present disclosure, the pharmaceutical composition for treating dermatitis of the present disclosure and/or a medicament prepared in the use of Adenostemma lavenia extract in the manufacture a medicament for treating dermatitis of the present disclosure may be administered to a subject in need thereof, but it is not limited thereto.

The Adenostemma lavenia extract of the present disclosure, the pharmaceutical composition for treating dermatitis of the present disclosure and/or a medicament prepared in the use of Adenostemma lavenia extract in the manufacture a medicament for treating dermatitis of the present disclosure may be administered parenterally, orally, by an inhalation spray, or via an implanted reservoir, but it is not limited thereto. The parenteral methods may comprise smearing the affected area, subcutaneous, intracutaneous, intravenous, intramuscular, intra-articular, intra-arterial, intrasynovial, intrasternal, intrathecal, and intralesional injection, as well as infusion techniques, etc.

Topically used form for smearing may comprise ointments, creams, solutions, gels, mousse, etc. but they are not limited thereto.

Moreover, the subject need to be administered the Adenostemma lavenia extract of the present disclosure, the pharmaceutical composition for treating dermatitis of the present disclosure and/or a medicament prepared in the use of Adenostemma lavenia extract in the manufacture a medicament for treating dermatitis of the present disclosure may comprise, but is not limited to, a vertebrate. Furthermore, the vertebrate mentioned above may comprise a fish, an amphibian, a reptile, a bird or a mammal, but it is not limited thereto. Examples of the mammal may comprise, but are not limited to a human, an orangutan, a monkey, a horse, a donkey, a dog, a cat, a rabbit, a guinea pig, a rat and a mouse. In one embodiment, the subject mentioned above may be a human.

In addition, the present disclosure may also provide a method for treating dermatitis. The method for treating dermatitis of the present disclosure may comprise, but is not limited to, administering an effective amount of Adenostemma lavenia extract or administering an effective amount of a pharmaceutical composition for treating dermatitis to an subject in need thereof.

Example of the aforementioned dermatitis may comprise, but is not limited to, atopic dermatitis, contact dermatitis, or any combination thereof. In one embodiment, the aforementioned dermatitis may be atopic dermatitis. In another embodiment, the aforementioned dermatitis may be contact dermatitis.

Furthermore, the Adenostemma lavenia extract used in the method for treating dermatitis of the present disclosure be any Adenostemma lavenia extracts of the present disclosure mentioned above. Similarly, the pharmaceutical composition for treating dermatitis used in the method for treating dermatitis of the present disclosure may be any pharmaceutical compositions for treating dermatitis of the present disclosure mentioned above.

Moreover, in the method for treating dermatitis of the present disclosure, the subject in need of the Adenostemma lavenia extract of the present disclosure or the pharmaceutical composition for treating dermatitis of the present disclosure may comprise, but is not limited to, a vertebrate. Furthermore, the vertebrate mentioned above may comprise a fish, an amphibian, a reptile, a bird or a mammal, but it is not limited thereto. Examples of the mammal may comprise, but are not limited to a human, an orangutan, a monkey, a horse, a donkey, a dog, a cat, a rabbit, a guinea pig, a rat and a mouse. In one embodiment, the subject may be a human.

### Examples

### A. Methods

### 1. Evaluation of IL-4 Secretion Inhibitory Activity by EL4 Cells

EL4 cells were seeded into a 96-well culture plate (1×10⁵ cells/well). Next, a sample to be tested with an appropriate concentration was added to the cells (10 μL/well) and mixed well. After that, the 96-well culture plate was then incubated in a 37° C., 5% CO₂ cell incubator for 2 hours. Stimulant A23187 and phorbol 12-myristate-13-acetate (PMA) were then added to the cells in the 96-well culture plate. After mixing well, the 96-well culture plate was incubated in the cell incubator for 18 hours.

After that, the above-mentioned 96-well culture plate was centrifuged at 1,200 rpm for 5 minutes, and the supernatant was collected to a new 96-well culture plate.

MTT was added to the cells remaining in the original 96-well culture plate, and then the original 96-well culture plate was placed in a 37° C., 5% CO₂ cell incubator for 1 hour. Afterwards, DMSO was added to the cells to dissolve out crystal violet. After mixing evenly, the absorbance at 570 nm of each well was measured to calculate the cell viability. In addition, the above-mentioned supernatant placed in a new 96-well plate was diluted, and the IL-4 content thereof was detected with the Mouse IL-4 ELISA kit (R&D systems, Mouse IL-4 DuoSet ELISA, Catalog #: DY404).

For all experimental results, the group only added with A23187 and PMA was used as 100% to calculate the inhibition rate of IL-4 secretion, and half-maximal inhibitory concentration (IC₅₀) and half-maximal cytotoxic concentration (CC₅₀) were further calculated.

### 2. Experiment for Animal Model of 2,4-Dinitrochlorobenzene (DNCB)-Induced Dermatitis

### 2.1 Experimental Animals

6-week-old BALB/c male mice were purchased from the National Laboratory Animal Center (Taipei City, Taiwan). After entering the general mouse experiment area of the Animal Experiment Station of the Industrial Technology Research Institute, the mice were marked, divided into cages, weighed and quarantined for a week.

During the quarantine period, the animals were acclimatized and observed for activity thereof. If the body weight of the animal increased normally, the animal was moved into the feeding area to wait for the experiment to be performed. In the rearing area, the illumination was set to a 12-hour light-dark cycle (7:30 am-7:30 pm is the light cycle), and the room temperature was set to 23±2° C. Animals were free to eat and drink. The rearing conditions were in line with national laboratory animal standards. There are abundant basic reference materials and data for this strain of experimental animals, and they are suitable for functional evaluation tests for atopic dermatitis and/or contact dermatitis. The experimental methods have been reviewed and approved by Institutional Animal Care and Use Committee (IACUC) of Industrial Technology Research Institute.

2.2 Method for Inducing Atopic Dermatitis in Mice with 2,4-Dinitrochlorobenzene

Before the experiment, the dorsal hair of BALB/c male mice was shaved with an electric shaver. On the first day (Day 1) of the experimental period, sensitization/stimulation was performed on skin with an area of about 2×3 cm² at the back of the mouse with 100 μL of 1% 2,4-dinitrochlorobenzene (prepared in a solution formed by mixing acetone and olive oil at a volume ratio of 4:1). After that, sensitization/stimulation was performed on dorsal skin and ear skin of the mouse respectively with 100 μL and 20 μL of 0.5% 2,4-dinitrochlorobenzene (prepared in a solution formed by mixing acetone and olive oil at a volume ratio of 4:1) with the frequency of twice a week and was lasted three weeks (Day 4-Day 28) to make the mouse to develop symptoms of atopic dermatitis.

Except the group without sensitization treatment (without any treatment (Naïve)), test sample or reference drug was orally administered to the mice in other groups every day from the 10^{th} day of the experimental period for three weeks (the 10^{th} day (Day 10)) to the 30^{th} day (Day 30)). Cyclosporine A (CsA) (Sandimmun, Capsules) was used as a reference drug in the animal model experiment of atopic dermatitis induced by 2,4-dinitrochlorobenzene, and was orally administered 30 mg/kg every day.

During the experimental period, photographs were taken regularly to record swelling and damage (skin lesions) situation of mouse skin. On the 31^{st} day of the experiment, animals were sacrificed with excess CO₂. The back and ear skin samples of the mice were collected to perform hematoxylin and eosin (H&E) staining to analyze the epidermis thickness and total histological score for the mice.

### 2.3 Skin Histopathological Analysis

The dorsal skin specimens of the mice collected upon sacrifice were placed on a small piece of paper to keep them flat. Along with the ear skin specimens, they were fixed in neutral formalin solution for their tissue shape and structure. Next, the fixed specimens were dehydrated and embedded with paraffin, and the completed paraffin specimens were cut into 3-4 μm paraffin sections with a microtome, and then stained with hematoxylin-eosin. The hematoxylin-eosin stained sections were measured for the epidermal thickness and evaluated for the severity of skin histopathology by professional pathologists according to the methods listed in the literature, Shackelford C, Long G, Wolf J, Okerberg C, Herbert R. Qualitative and quantitative analysis of nonneoplastic lesions in toxicology studies. Toxicologic pathology. 2002; 30(1):93-6.

The histopathological grading standards are as follows: Grade 0=not present; Grade 1=minimal (<1%); Grade 2=slight (1-25%); Grade 3=moderate (26-50%); Grade 4=moderately severe (51-75%); Grade 5=severe/high (76-100%).

### 2.4 Data Analysis

The data in this experiment are expressed as standard error of the mean (SEM)=mean±standard error. All groups were mainly compared with the group administered with the vehicle. Depending on the nature of the data, one-way ANOVA or two-way ANOVA combined with Dunnett's multiple comparisons test, Student's t test or Mann-Whitney test was used to calculate whether there is a difference. When the p value is less than 0.05, it represents that there is a statistically significant difference between the groups.

### 3. Experiment of Atopic Dermatitis Model in NC/Nga Mice

### 3.1 Experimental Animals

6-week-old NC/NgaTndCrlj male mice were purchased from Charles River Japan (Tokyo, Japan). After entering the general mouse experiment area of the Animal Experiment Station of the Industrial Technology Research Institute, the mice were marked, divided into cages, weighed and quarantined for a week.

During the quarantine period, the animals were acclimatized and observed for activity thereof. If the body weight of the animal increased normally, the animal was moved into the feeding area to wait for the experiment to be performed. In the rearing area, the illumination was set to a 12-hour light-dark cycle (7:30 am-7:30 pm is the light cycle), and the room temperature was set to 23±2° C. Animals were free to eat and drink. The rearing conditions were in line with national laboratory animal standards. There are abundant basic reference materials and data for this strain of experimental animals, and they are suitable for functional evaluation tests for atopic dermatitis. The experimental methods have been reviewed and approved by Institutional Animal Care and Use Committee (IACUC) of Industrial Technology Research Institute.

### 3.2. Method for Inducing Atopic Dermatitis in NC/Nga Mice

When experiment began, NC/Nga mice aged approximately 10-12 weeks were S-type grouped according to body weight to make each group mean body weight and body weight distribution trend similar.

The dorsal hair of the experimental animals was shaved one day before the experiment was started, and the area of hair shaved was about 4×2 cm². On the first day of the experiment (Day 0), 150 μL and 20 μL of 4% sodium dodecyl sulfate (SDS) were administered to the dorsal skin and ear skin of the mice by skin smearing, respectively. After 3 hours, 100 mg and 10 mg of Dermatophagoides farina body (Dfb) ointment (Biostir AD) (purchased from Biostir (Osaka, Japan)) were administered in the same manner to perform sensitization/stimulation on dorsal skin and ear skin of the mice. The frequency of sensitization/stimulation was 2 times a week for 6 consecutive weeks.

Test samples or solvents were administered according to the experimental design, and the volume of administration was 10 mL/kg. F4AB was orally administered at 30, 100, 200 or 400 mg/kg every day. A JAK inhibitor, upadacitinib (purchased from MedChemExpress (Monmouth Junction, NJ, USA)), was used as a reference drug, and was orally administered at 1, 3 or 10 mg/kg every day. Animals were given test drugs after sensitization/stimulation on Day 0, and the clinical observation scoring for the dorsal skin was performed on Day 0, the 7^{th} day (Day 7), the 14^{th} (Day 14), the 21^{st} (Day 21), the 28^{th} day (Day 28), the 35^{th} day (Day 35) and the 42^{nd} day (Day 42). On the 42^{nd} day of the experiment (Day 42), the animals were sacrificed, the blood was collected to perform IgE analysis, and the dorsal skin tissue was taken to perform pathological analysis.

### 3.3 Observation of Skin Clinical Symptoms

The skin clinical observation scoring of atopic dermatitis in NC/Nga mice induced by Dermatophagoides farina body is aimed at skin damage symptoms of erythema/hemorrhage, scarring/dryness, edema, excoriation/erosion, etc., occurring on the dorsal skin to perform observation, evaluation and record respectively. The scaling standards of the above clinical symptoms are as follows: Scale 0, no symptoms; Scale 1, mild; Scale 2, moderate; Scale 3, severe. The total score of clinical severity of dermatitis (Score of Skin Lesion) is the sum of the scores of the above-mentioned clinical symptom scoring items.

### 3.4 Skin Histopathological Analysis

The dorsal skin specimens of the mice collected upon sacrifice were placed on a small piece of paper to keep them flat. Along with the ear skin specimens, they were fixed in neutral formalin solution for their tissue shape and structure. Next, the fixed specimens were dehydrated and embedded with paraffin, and the completed paraffin specimens were cut into 3-4 μm paraffin sections with a microtome, and then stained with hematoxylin-eosin. The hematoxylin-eosin stained sections were measured for the epidermal thickness and evaluated for items such as the severity of skin histopathology, inflammation, necrosis, hyperplasia, hyperkeratosis, by professional pathologists according to the methods listed in the literature, Shackelford C, Long G, Wolf J, Okerberg C, Herbert R. Qualitative and quantitative analysis of nonneoplastic lesions in toxicology studies. Toxicologic pathology. 2002; 30(1):93-6.

The histopathological grading standards are as follows: Grade 0=not present; Grade 1=minimal (<1%); Grade 2=slight (1-25%); Grade 3=moderate (26-50%); Grade 4=moderately severe (51-75%); Grade 5=severe/high (76-100%).

### 3.5 Data Analysis

The data in this experiment are expressed as standard error of the mean (SEM)=mean±standard error. All groups were mainly compared with the group administered with the vehicle. Depending on the nature of the data, one-way ANOVA or two-way ANOVA combined with Dunnett's multiple comparisons test, Student's t test or Mann-Whitney test was used to calculate whether there is a difference. When the p value is less than 0.05, it represents that there is a statistically significant difference between the groups.

B. Preparation of Adenostemma lavenia Extract
1. Preparation of Ethanol Extract of Adenostemma lavenia
1.1 Preparation of Adenostemma lavenia Extract 0805

First, dried leaves of Adenostemma lavenia were pulverized to obtain a plant material of Adenostemma lavenia.

Next, the above-mentioned plant material of Adenostemma lavenia was extracted with 8 times its weight of 95% ethanol of at 60° C. for 8 hours, and then filtered to obtain the extract solution of the first extraction and the raffinate of the plant material of Adenostemma lavenia.

After that, the foregoing raffinate of the plant material of Adenostemma lavenia was extracted again with 8 times its weight of 95% ethanol of at 60° C. for 8 hours, and then filtered to obtain the extract solution of the second extraction and the raffinate of the plant material of Adenostemma lavenia.

The extract solutions of the two extractions were combined to obtain a combined extract solution. Finally, the combined extract solution was concentrated under reduced pressure in a rotary evaporator to remove the solvent to obtain an extract, which was named Adenostemma lavenia extract 0805.

1.2 Preparation of Adenostemma lavenia Extract 0805-F

50 g of the Adenostemma lavenia extract 0805 obtained above was heated at 50° C. with 8 times its weight of 95% ethanol to dissolve it to obtain a mixture solution.

5 times weight of silica gel (250 g) was added to this mixture solution for immobilization, and a mixed solvent of n-hexane/ethyl acetate was used as a mobile phase to perform column chromatography (glass column with an inner diameter of 10 cm, filled with 2 kg silica gel).

6 fractions were obtained in column chromatography, and named as 0805-f1, 0805-f2, 0805-f3, 0805-f4, 0805-f5 and 0805-f6, sequentially.

After that, based on the method described in “1. Evaluation of IL-4 secretion inhibitory activity by EL4 cells” of “A. Methods” above, IL-4 secretion inhibitory activities of the Adenostemma lavenia extract 0805 and its 6 fractions were evaluated by EL4 cells. The results are shown in Table 1.

TABLE 1
Evaluation results of IL-4 secretion inhibitory activities of the
Adenostemma lavenia extract 0805 and its 6 fractions

| Test sample | IC₅₀ (μg/mL) | CC₅₀ (μg/mL) |
|---|---|---|
| 0805 | 55.4 | >100 |
| 0805-f1 | >50 | >50 |
| 0805-f2 | >50 | >50 |
| 0805-f3 | 4.6 ± 1.2 | 30.9 ± 14.8 |
| 0805-f4 | 4.4 ± 0.5 | 38.3 ± 7.7 |
| 0805-f5 | 45.6 ± 6.2 | >50 |
| 0805-f6 | >50 | >50 |

Table 1 shows that Adenostemma lavenia extract 0805 is capable of inhibiting IL-4 secretion (IC₅₀=55.4 μg/mL). Among the aforementioned 6 fractions, 0805-f3 and 0805-f4 had better IL-4 secretion inhibitory activities, with IC₅₀ of 4.6±1.2 μg/mL and 4.4±0.5 μg/mL, respectively. Therefore, fraction 0805-f3 and fraction 0805-f4 were combined to obtain a combined fraction.

Afterwards, the combined fraction was concentrated under reduced pressure with a rotary evaporator to remove the solvent, and a product was obtained, which was named Adenostemma lavenia extract 0805-F.

2. Preparation of Water Extract of Adenostemma lavenia
2.1 Preparation of Adenostemma lavenia Extract W

First, leaves of Adenostemma lavenia was pulverized, and then put in a filter bag to obtain a plant material of Adenostemma lavenia.

Next, the above-mentioned plant material of Adenostemma lavenia was extracted in a pressurized reaction tank with 30 times its weight of pure water (extraction condition: 102.97 kPa; 120° C.; 20 minutes) to obtain an extract, and the extract was taken out from the pressurized reaction tank, and the above-mentioned plant material of Adenostemma lavenia was repeatedly extracted under the same conditions. A total of three extractions were performed. The extracts obtained from the three extractions were combined to obtain a combined extract and named Adenostemma lavenia extract W.

2.2 Preparation of Adenostemma lavenia Extract F4AB

### Column Chromatography I:

The aforementioned Adenostemma lavenia extract W was loaded into a column (12 inches in diameter, containing 100 L of HP20 resin). After that, the column was eluted with pure water (200 L) and the eluate was discarded, and then the column was eluted with 50% ethanol aqueous solution (200 L) and the eluate was discarded. Then, the column was eluted with 75% ethanol aqueous solution (200 L) and the eluate was collected as a preliminary purified extract solution.

The preliminary purification extract was further concentrated to obtain a concentrated preliminary purified extract solution.

### Column Chromatography II:

After the concentrated preliminary purified extract solution was fixed with the silica gel of 5 times of weight of its solid content weight, silica gel column (a column with a diameter of 10 cm filled with the silica gel of 20 times of weight of solid content weight of the concentrated preliminary purified extract solution) was used for column chromatography with n-hexane/ethyl acetate (1:1) as a mobile phase. Fractions were collected in section.

According to the method described in “1. Evaluation of IL-4 secretion inhibitory activity by EL4 cells” of “A. Methods” above, IL-4 secretion inhibitory activities of all fractions obtained in the column chromatography were evaluated by EL4 cells, and the fractions with IL-4 secretion inhibitory activities were combined to obtain an active fraction.

The aforementioned activity fraction was further concentrated to obtain a concentrated active fraction.

The above-mentioned concentrated active fraction was heated and dissolved with 4 times its weight of 95% aqueous ethanol solution at 60° C. to form an active solution.

The aforementioned active solution was left to stand for 24 hours to make crystals form therein. Finally the crystals were collected and dried. The obtained dried crystals were named Adenostemma lavenia extract F4AB.

C. Animal Experiment for Adenostemma lavenia Extract 0805

According to the method described in “2. Experiment for animal model of 2,4-dinitrochlorobenzene (DNCB)-induced dermatitis” of “A. Methods” above, animal experiment was performed for Adenostemma lavenia extract 0805.

After the mice were sensitized with 2,4-dinitrochlorobenzene, 300 mg/kg of the Adenostemma lavenia extract 0805 was orally administered every day from the 10^{th} day of the experiment for three consecutive weeks (the 10^{th} day (Day 10) to the 30^{th} day (Day 30)).

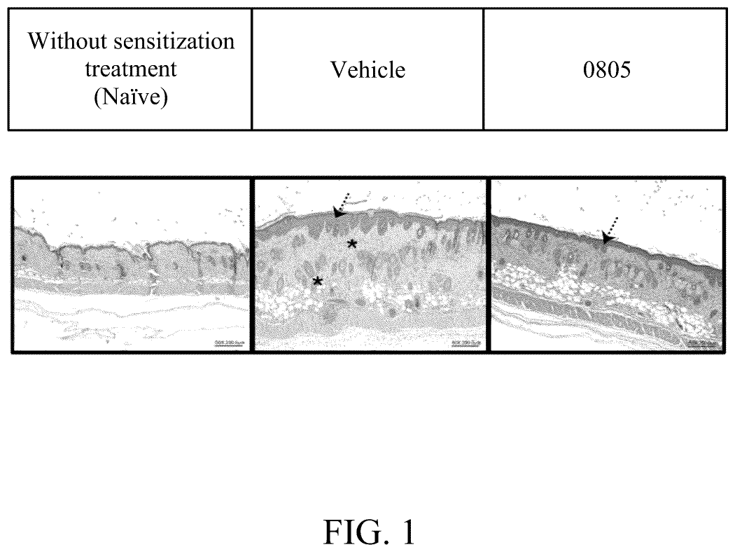

On the 31^{st} day of the experiment, animals were sacrificed with excessive CO₂, and then mouse skin tissue section staining and pathological section analysis, epidermal thickness measurement and total skin pathological severity scoring were performed. The experimental results are shown in FIGS. 1 to 3.

FIG. 1 shows the hematoxylin-eosin staining results of the dorsal skin tissue sections of the mice in the group without sensitization treatment (Naïve), the group administered with the vehicle and the group administered with Adenostemma lavenia extract 0805. Position marked by an arrow indicates hyperplasia and position marked by * indicates inflammation.

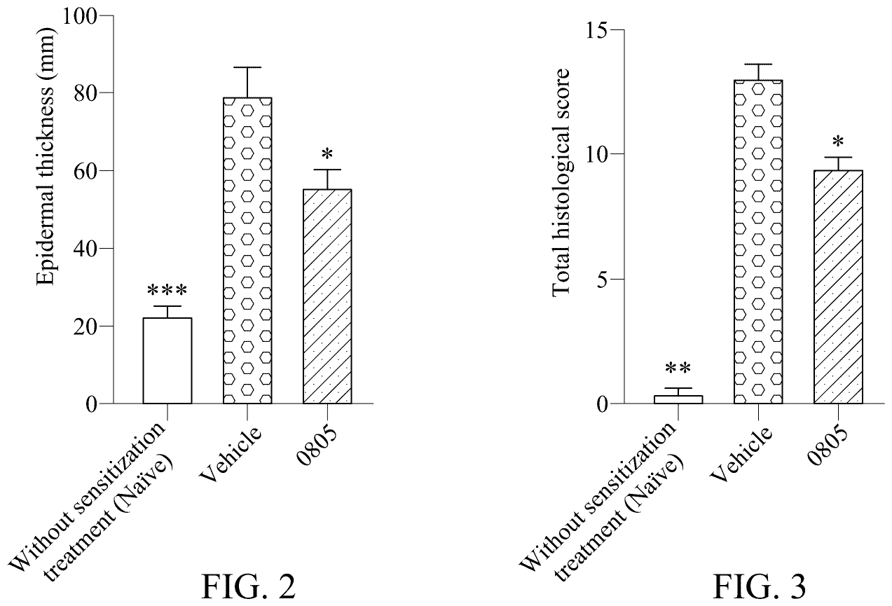

FIG. 2 shows epidermal thickness of the dorsal skin of the mice in the group without sensitization treatment (Naïve), the group administered with the vehicle and the group administered with Adenostemma lavenia extract 0805, and FIG. 3 shows total histological scores for the dorsal skin of the mice in the group without sensitization treatment (Naïve), the group administered with the vehicle and the group administered with Adenostemma lavenia extract 0805. Data are presented as mean±standard error (Mean±SEM) (n=5). Student's t-test (epidermal thickness measurement) or Mann-Whitney test (histopathological scoring) was used for statistical analysis. Compared to the group administered with the vehicle, p<0.05 represents that there is a statistically significant difference, *p<0.05; **p<0.01; ***p<0.001.

Based on the experimental results shown in FIGS. 1 to 3, it is known that Adenostemma lavenia extract 0805 is capable of significantly reducing the hyperplasia of the dorsal epidermis and the total pathological damage of the dorsal skin.

D. Animal Experiments of Adenostemma lavenia Extract 0805-F

According to the method described in “2. Experiment for animal model of 2,4-dinitrochlorobenzene (DNCB)-induced dermatitis” of “A. Methods” above, animal experiment was performed for Adenostemma lavenia extract 0805-F.

After the mice were sensitized with 2,4-dinitrochlorobenzene, 300 mg/kg of the Adenostemma lavenia extract 0805-F or 30 mg/kg of the reference drug, cyclosporine A, was orally administered every day from the 10^{th} day of the experiment for three consecutive weeks (the 10^{th} day (Day 10) to the 30^{th} day (Day 30)).

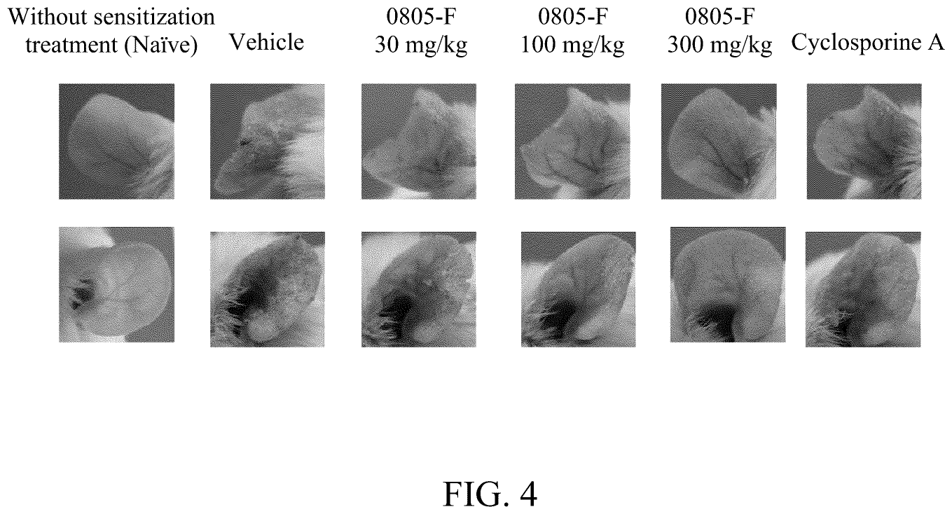

During the experiment, the mouse ears were photographed to record the skin swelling and damage (skin lesions) condition of the mouse ears and thickness of the mouse ears were measured. The photographs of mouse ears on the 30^{th} day of the experiment are shown in FIG. 4.

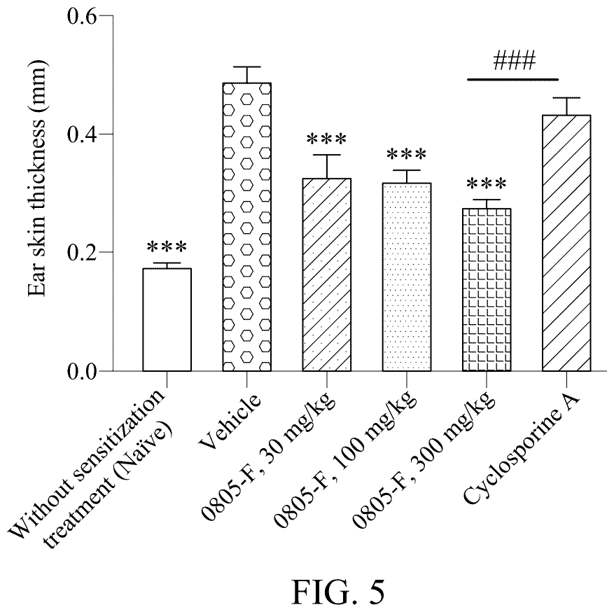

Moreover, the measurement results of the mouse ear thickness on the 30^{th} day of the experiment are shown in FIG. 5. Data are presented as mean±standard error (Mean±SEM) (n=5). In FIG. 5, for the comparisons of the ear thickness of respective groups to the ear thickness of the group administered with the vehicle, statistical analysis was performed by two-way ANOVA with Dunnett's multiple comparisons test. p<0.05 represents that there is a statistically significant difference, ***p<0.001; in FIG. 5, for the comparisons of the ear thickness of the groups administered with 300 mg/kg of Adenostemma lavenia extract 0805-F to the ear thickness of the group administered with cyclosporine A, statistical analysis was performed by Student's t-test. p<0.05 represents that there is a statistically significant difference, ###p<0.001.

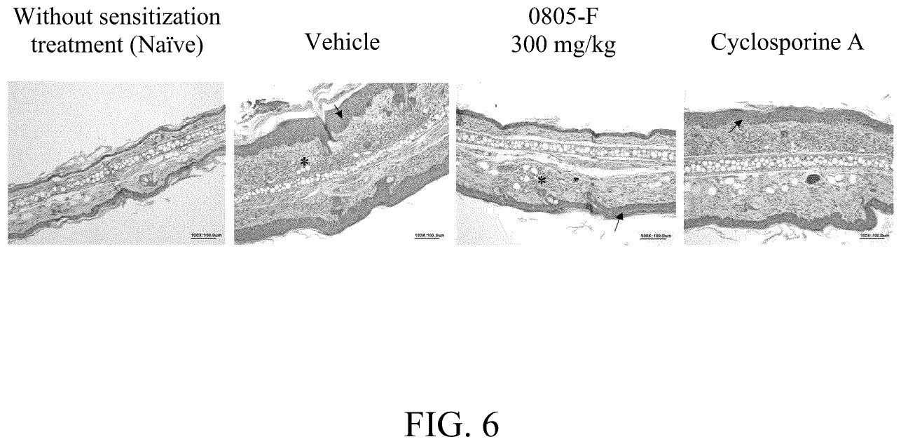

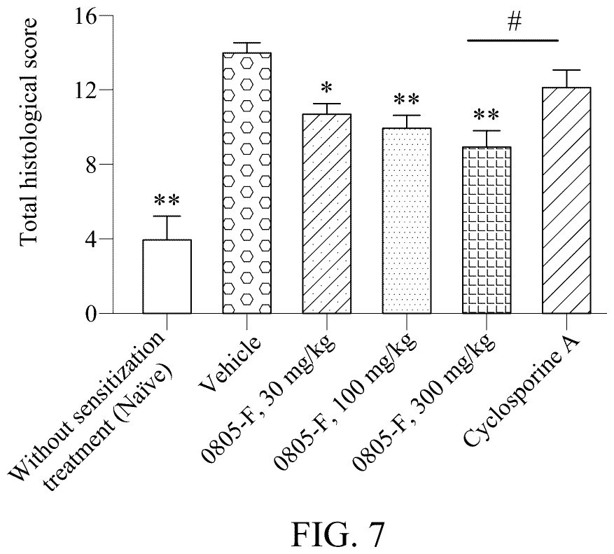

On the 31^{st} day of the experiment, animals were sacrificed with excessive CO₂, and then mouse ear skin tissue section staining and pathological section analysis, and scoring for total pathological severity of ear skin were performed. The experimental results are shown in FIGS. 6 and 7.

FIG. 6 shows the hematoxylin-eosin staining results of ear skin tissue sections of the mice in the group without sensitization treatment (Naïve), the group administered with the vehicle, the group administered with Adenostemma lavenia extract 0805-F and the group administered with cyclosporine A. Position marked by an arrow indicates hyperplasia and position marked by * indicates inflammation.

FIG. 7 shows the total histological score of ear skin of mice in the non-sensitization treatment group, vehicle administration group, Adenostemma lavenia extract 0805-F administration group and cyclosporine A administration group. Data are presented as mean±standard error (Mean±SEM) (n=5). For the comparisons of the total histological score of respective groups with the total histological score of the group administered with the vehicle, statistical analysis was performed by Mann-Whitney test. p<0.05 represents that there is a statistically significant difference, *p<0.05; **p<0.01. For the comparisons of the total histological score of the group administered with 300 mg/kg of Adenostemma lavenia extract 0805-F to the total histological score of the group administered with cyclosporine A, statistical analysis was performed by Mann-Whitney test. p<0.05 represents that there is a statistically significant difference, #p<0.05.

According to the experimental results shown in FIG. 4 to FIG. 7, Adenostemma lavenia extract 0805-F is capable of significantly improving the clinical appearance, pathological symptoms and ear thickness of mouse ear skin, and presents a dose effect, and the efficacy thereof is better than the clinical drug, cyclosporine A.

E. Animal Experiments for Adenostemma lavenia Extract F4AB

According to the method described in “Experiment of atopic dermatitis model in NC/Nga mice” of “A. Methods” above, animal experiment was performed for Adenostemma lavenia extract F4AB.

F4AB is orally administered every day at 30, 100, 200 or 400 mg/kg. A JAK inhibitor, upadacitinib, was used as a reference drug, and orally administered at 1, 3 or 10 mg/kg every day. Animals were given test drugs after sensitization/stimulation on Day 0, and the clinical observation scoring for the dorsal skin was performed on Day 0, the 7^{th} day (Day 7), the 14^{th} (Day 14), the 21^{st} (Day 21), the 28^{th} day (Day 28), the 35^{th} day (Day 35) and the 42^{nd} day (Day 42). On the 42^{nd} day of the experiment (Day 42), the animals were sacrificed, the blood was collected to perform IgE analysis, and the dorsal skin tissue was taken to perform pathological analysis.

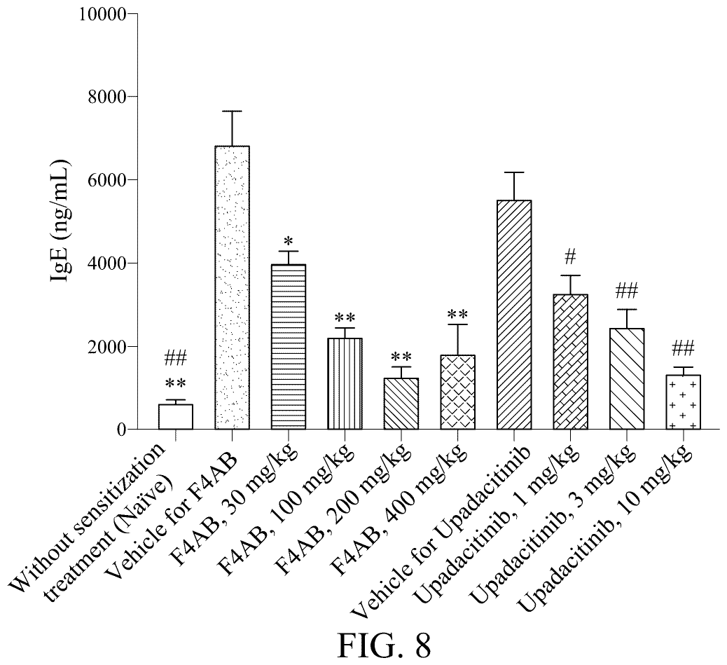

The measurement result of mouse serum IgE concentration of each group is shown in FIG. 8. In FIG. 8, the data are presented as mean±standard error (Mean±SEM) (n=5), and statistical analysis was performed by Student's t-test. For comparison of the group administered with Adenostemma lavenia extract F4AB to the group administered with its vehicle, p<0.05 represents that there is a statistically significant difference, *p<0.05; **p<0.01. For comparison of the group administered with upadacitinib to the group administered with its vehicle, p<0.05 represents that there is a statistically significant difference, #p<0.05; ##p<0.01.

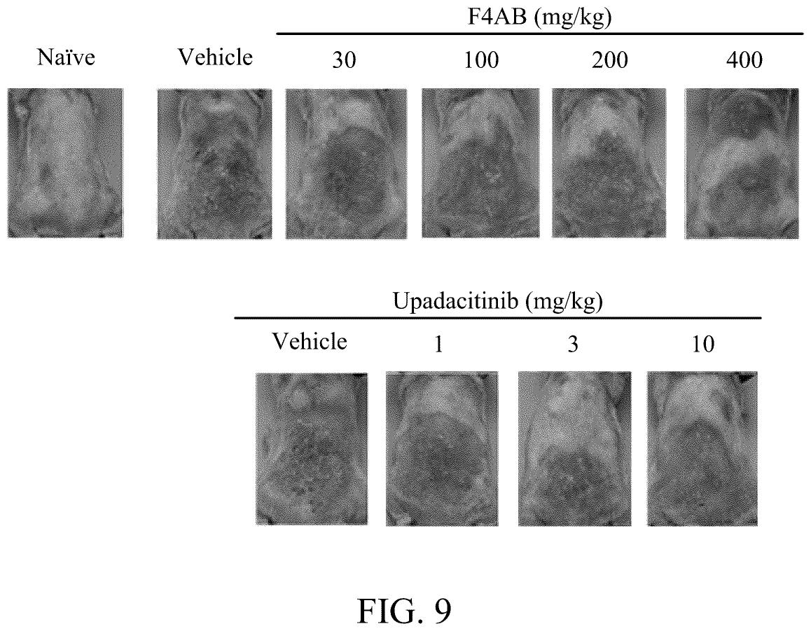

On the 42^{nd} day of the experiment, before the mice were sacrificed, their backs were photographed to record desquamation and damage (skin lesions) of the skin appearance. The results are shown in FIG. 9.

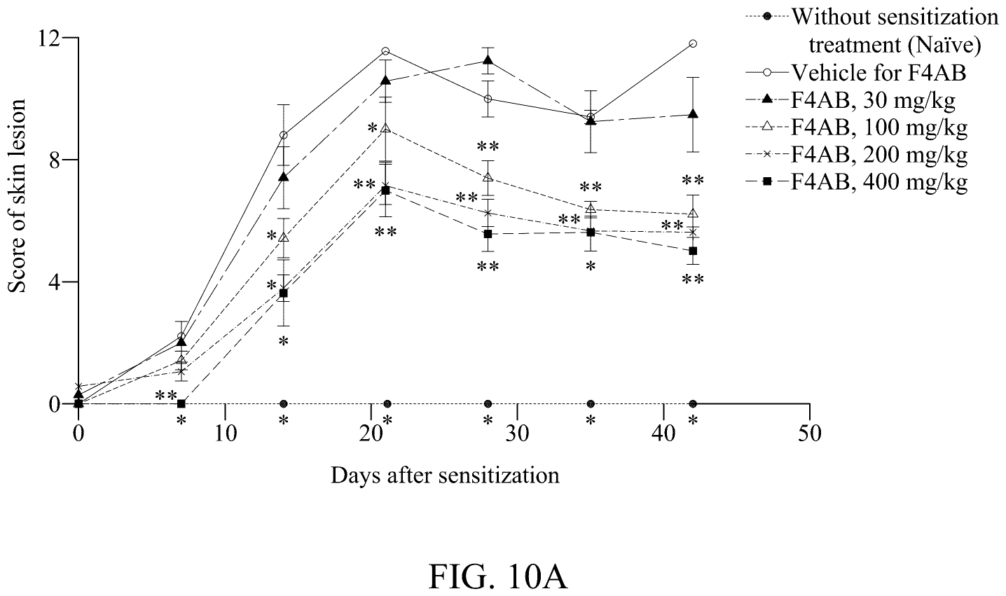

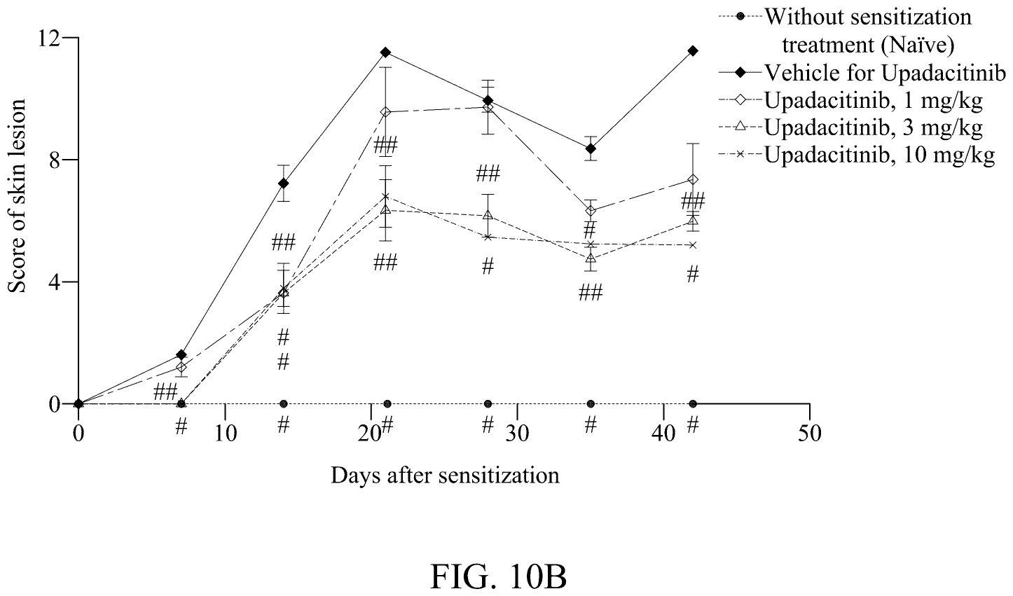

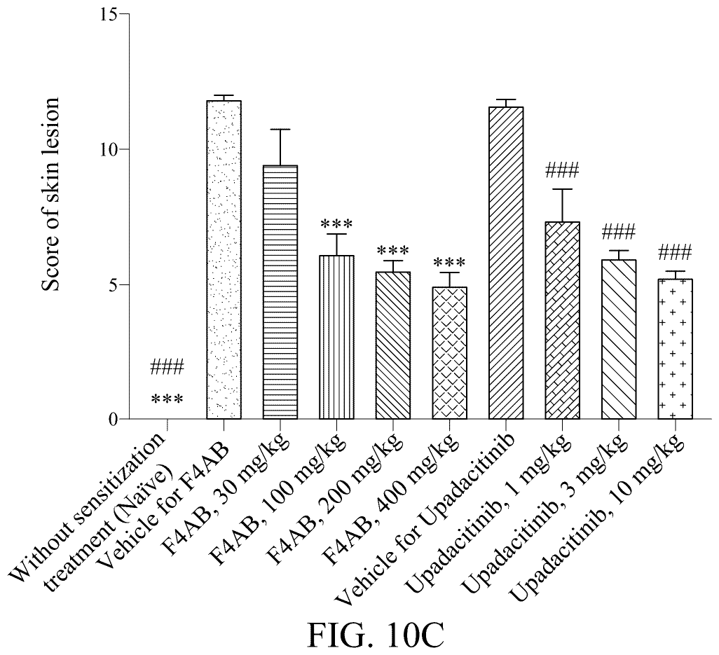

The total dermatitis clinical severity scoring (Score of Skin Lesion) was performed on the back of the mice every week during the experiment, and the results are shown in FIG. 10A and FIG. 10B. FIG. 10A shows the total dermatitis clinical severity score of the back of the mice administered with Adenostemma lavenia extract F4AB. The data are presented as mean±standard error (Mean±SEM) (n=5), and statistical analysis is performed by Mann-Whitney test. Compared to the group administered with its vehicle, p<0.05 represents that there is a statistically significant difference, *p<0.05; **p<0.01. FIG. 10B shows the total dermatitis clinical severity score of the back of mice administered with upadacitinib. The data are presented as mean±standard error (Mean±SEM) (n=5), and statistical analysis is performed by Mann-Whitney test. Compared to the group administered with its vehicle, p<0.05 represents that there is a statistically significant difference, #p<0.05; ##p<0.01.

On the 42^{nd} day of the experiment, before the mice were sacrificed, the total dermatitis clinical severity scoring (Score of Skin Lesion) was performed on the back of the mice, and the results are shown in FIG. 10C. Data are presented as mean±standard error (Mean±SEM) (n=5), and statistical analysis was performed by Mann-Whitney test. For comparison of the group administered with Adenostemma lavenia extract F4AB to the group administered with its vehicle, p<0.05 represents that there is a statistically significant difference, ***p<0.001. For comparison of the group administered with upadacitinib to the group administered with its vehicle, p<0.05 represents that there is a statistically significant difference, ###p<0.001.

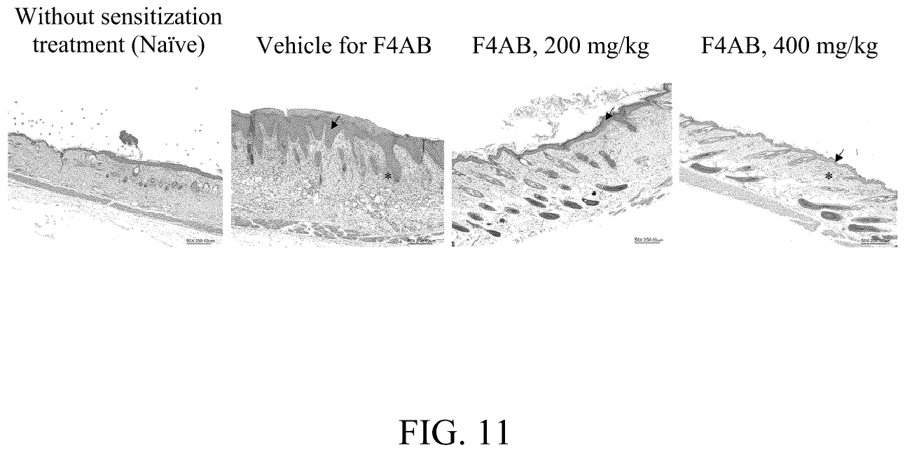

After the mice were sacrificed, skin tissue section staining, epidermis thickness measurement and pathological section analysis were performed on the mice. The experimental results are shown in FIGS. 11, 12, 13 and 14A to 14E.

FIG. 11 shows the hematoxylin-eosin staining results of the dorsal skin tissue sections of mice in the group without sensitization treatment, the group administered with Adenostemma lavenia extract F4AB, and the group administered with the vehicle. Position marked by an arrow indicates hyperplasia and position marked by * indicates inflammation.

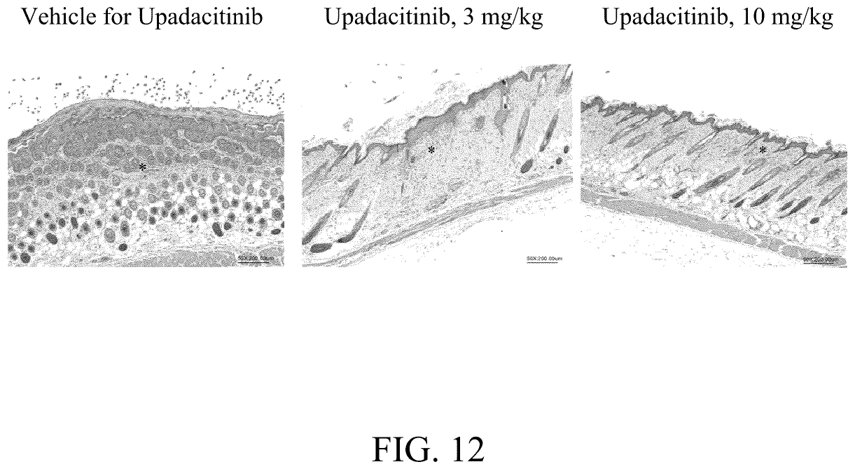

FIG. 12 shows the hematoxylin-eosin staining results of the dorsal skin tissue sections of the mice in the group without sensitization treatment, the group administered with upadacitinib, and the group administered with the vehicle. Position marked by * indicates inflammation.

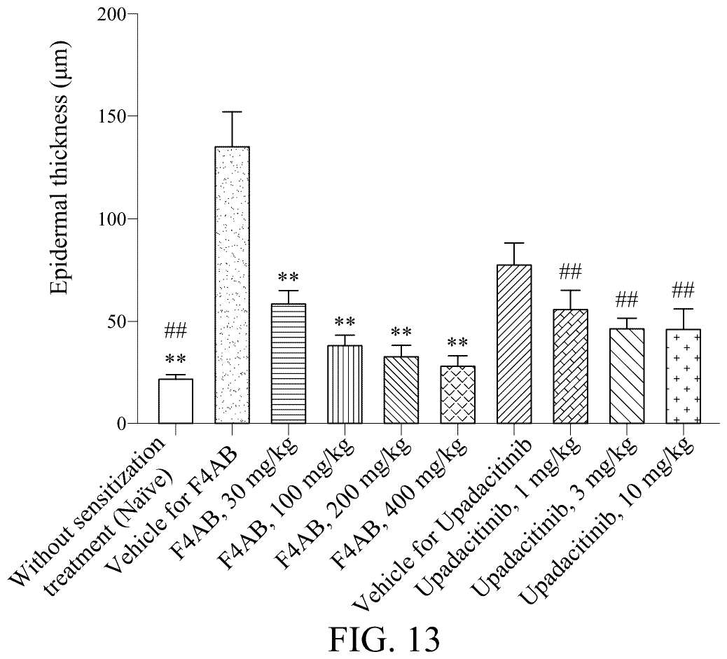

FIG. 13 shows epidermal thickness of the dorsal skin of the mice in the group without sensitization treatment, the group administered with the vehicle for Adenostemma lavenia, the group administered with Adenostemma lavenia extract F4AB, the group administered with the vehicle for upadacitinib, the group administered with upadacitinib. Data are presented as mean #standard error (Mean±SEM) (n=5), and statistical analysis was performed by Student's t-test. For comparison of the group administered with represents that there is a statistically significant difference, **p<0.01. For comparison of the group administered with upadacitinib to the group administered with its vehicle, p<0.05 represents that there is a statistically significant difference, ##p<0.01.

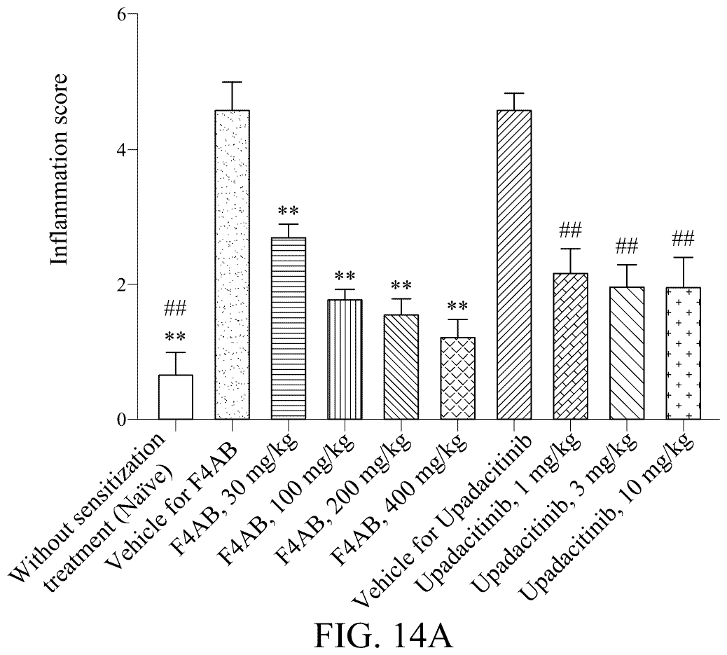

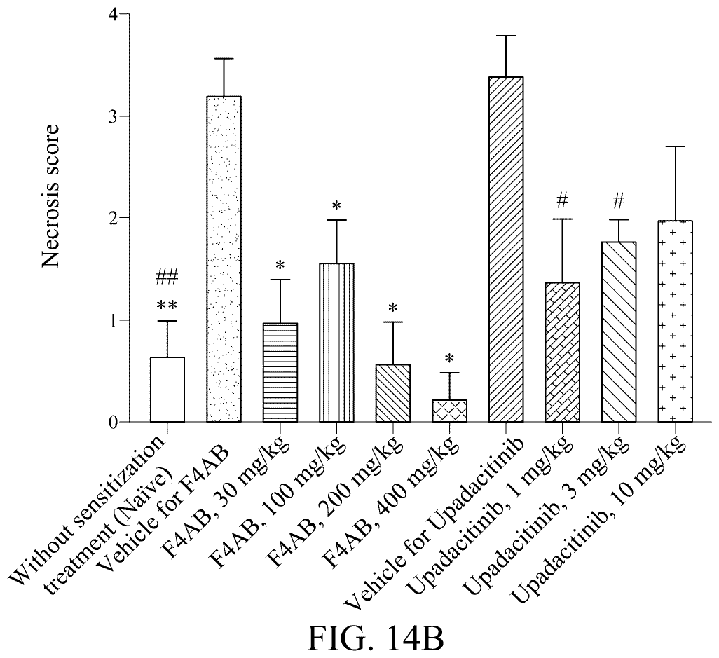

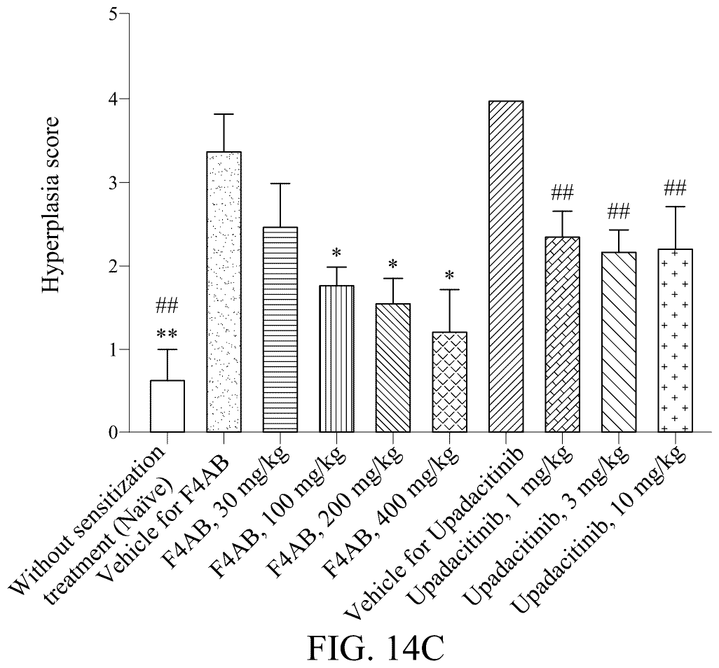

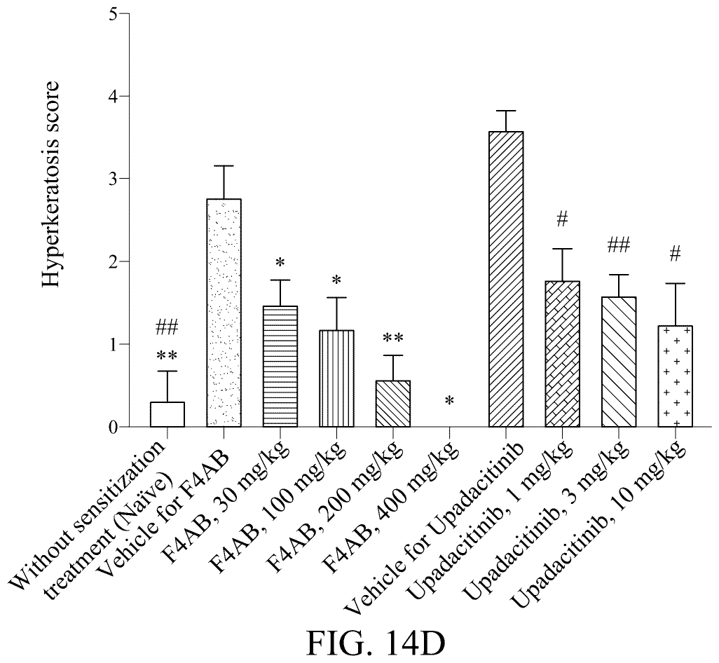

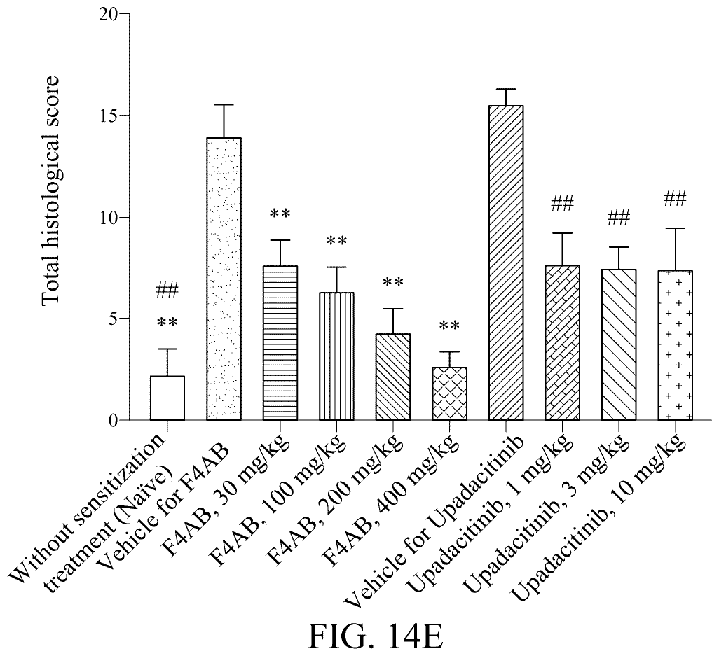

FIG. 14A to FIG. 14E respectively show the inflammation score, necrosis score, hyperplasia score, hyperkeratosis score and skin total histopathological severity score in the histopathological section analysis for the dorsal skin tissue of the mice. Data are presented as mean±standard error (Mean±SEM) (n=5), and statistical analysis was performed by Mann-Whitney test. For comparison of the group administered with Adenostemma lavenia extract F4AB to the group administered with its vehicle, p<0.05 represents that there is a statistically significant difference, *p<0.05; **p<0.01. For comparison of the group administered with upadacitinib to the group administered with its vehicle, p<0.05 represents that there is a statistically significant difference, #p<0.05; ##p<0.01.

According to the above experimental results, compared to the group administered with vehicle, the blood IgE concentration of mice in the group administered with 30-400 mg/kg Adenostemma lavenia extract F4AB is significantly reduced, while the group administered with the reference drug, 1-10 mg/kg upadacitinib, has the same effect.

In terms of the effect on the dorsal skin of mice, oral administration of Adenostemma lavenia extract F4AB and upadacitinib were both capable of significantly improving the clinical appearance and skin damage of the dorsal skin during the experiment, and the significant improvement and dose effect were still maintained on the day of sacrifice. In terms of histopathological analysis, oral administration of 30-400 mg/kg Adenostemma lavenia extract F4AB and 1-10 mg/kg upadacitinib were both capable of significantly improving the epidermal thickness of the dorsal skin, as well as reducing inflammation, hyperkeratosis, hyperplasia, necrosis and the total pathological damage.

All the above experiments have clearly proved that the Adenostemma lavenia extract of the present disclosure is able to be effectively used in the treatment of dermatitis, especially atopic dermatitis and contact dermatitis.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A method for treating dermatitis, comprising:
administering an effective amount of an Adenostemma lavenia extract or administering an effective amount of a pharmaceutical composition for treating dermatitis comprising an Adenostemma lavenia extract to a subject in need thereof,
wherein the dermatitis comprises atopic dermatitis or contact dermatitis.

2. The method for treating dermatitis as claimed in claim 1, wherein the Adenostemma lavenia extract is an ethanol extract of Adenostemma lavenia.

3. The method for treating dermatitis as claimed in claim 2, wherein the ethanol extract of Adenostemma lavenia is obtained by an extraction method, and the extraction method comprises:
(a) providing a plant material of Adenostemma lavenia; and
(b) performing a hot extraction procedure on the plant material of Adenostemma lavenia, and then performing a solid-liquid separation procedure to obtain an extract solution and produce a raffinate of the plant material of Adenostemma lavenia, wherein in the hot extraction procedure, a first ethanol solvent is used as an extraction solvent, and the weight ratio of the first ethanol solvent to the plant material of Adenostemma lavenia is 5-15:1.

4. The method for treating dermatitis as claimed in claim 3, wherein in step (b), the hot extraction procedure is performed at 40-80° C. for 1-15 hours.

5. The method for treating dermatitis as claimed in claim 3, wherein the extraction method further comprises:
(c) using the raffinate of the plant material of Adenostemma lavenia as the plant material of Adenostemma lavenia, and repeating step (b) at least one time, and combining all the obtained extract solutions to obtain a combined extract solution.

6. The method for treating dermatitis as claimed in claim 5, wherein the extraction method further comprises:
(d) performing a drying procedure on the combined extract solution to obtain a dry extract.

7. The method for treating dermatitis as claimed in claim 6, wherein the extraction method further comprises:
(e) performing a heating procedure on the dry extract in a second ethanol solvent until the dry extract is dissolved to form a mixture, wherein the weight ratio of the second ethanol solvent to the dry extract is 5-15:1; and
(f) performing column chromatography on the mixture, and collecting, or collecting and combining at least one fraction having an effect of inhibiting cell secretion of IL-4 as an active fraction, wherein in the column chromatography, a mixed solvent of n-hexane/ethyl acetate is used as a mobile phase.

8. The method for treating dermatitis as claimed in claim 7, wherein the heating procedure is performed at 30-60° C.

9. The method for treating dermatitis as claimed in claim 3, wherein the plant material of Adenostemma lavenia is from at least one of the following parts of Adenostemma lavenia:
a root, a stem, a leaf and a flower.

10. The method for treating dermatitis as claimed in claim 1, wherein the Adenostemma lavenia extract is a water extract of Adenostemma lavenia.

11. The method for treating dermatitis as claimed in claim 10, wherein the water extract of Adenostemma lavenia is obtained by an extraction method, and the extraction method comprises:
(a) providing a plant material of Adenostemma lavenia; and
(b) performing a pressurized extraction procedure on the plant material of Adenostemma lavenia, and then performing a solid-liquid separation procedure to obtain an extract solution and produce a raffinate of the plant material of Adenostemma lavenia, wherein in the pressurized extraction procedure, water is used as an extraction solvent, and the weight ratio of the water to the plant material of Adenostemma lavenia is 10-50:1.

12. The method for treating dermatitis as claimed in claim 11, wherein in step (b), the pressurized extraction procedure is performed at 101.4-140.0 kPa and 100-140° C. for 5-60 minutes.

13. The method for treating dermatitis as claimed in claim 11, wherein the extraction method further comprises:
(c) using the raffinate of the plant material of Adenostemma lavenia as the plant material of Adenostemma lavenia, and repeating step (b) at least one time, and combining all the obtained extract solutions to obtain a combined extract solution.

14. The method for treating dermatitis as claimed in claim 13, wherein the extraction method further comprises (d) performing a first column chromatography on the combined extract solution, and the first column chromatography comprises:
(d1) filling the combined extract solution into a column packed with resin;
(d2) after step (d1), eluting the column with water, and discarding eluate that flows out from the column;
(d3) after step (d2), eluting the column with a third ethanol solvent, and discarding the eluate that flows out from the column; and
(d4) after step (d3), eluting the column with a fourth ethanol solvent, and collecting the eluate that flows out from the column as a preliminary purified extract solution.

15. The method for treating dermatitis as claimed in claim 14, wherein the extraction method further comprises:
(e) performing a second column chromatography on the preliminary purified extract solution, and collecting, or collecting and combining at least one fraction having an effect of inhibiting cell secretion of IL-4 as an active fraction, wherein in the second column chromatography, a mixed solvent of n-hexane/ethyl acetate is used as a mobile phase.

16. The method for treating dermatitis as claimed in claim 15, wherein the extraction method further comprises concentrating the active fraction to obtain a concentrated active fraction.

17. The method for treating dermatitis as claimed in claim 16, wherein the extraction method further comprises:
performing a heating and dissolving procedure on the concentrated active fraction with a fifth ethanol solvent to form an active solution;
standing the active solution to make crystals form therein; and
collecting the crystals.

18. The method for treating dermatitis as claimed in claim 11, wherein the plant material of Adenostemma lavenia is from at least one of the following parts of Adenostemma lavenia:
a root, a stem, a leaf and a flower.

19. A pharmaceutical composition for treating dermatitis, comprising:
an Adenostemma lavenia extract; and
a pharmaceutically acceptable carrier or salt.

20. The pharmaceutical composition for treating dermatitis as claimed in claim 19, wherein the Adenostemma lavenia extract is an ethanol extract of Adenostemma lavenia.

21. The pharmaceutical composition for treating dermatitis as claimed in claim 20, wherein the ethanol extract of Adenostemma lavenia is obtained by an extraction method, and the extraction method comprises:
(a) providing a plant material of Adenostemma lavenia;
(b) performing a hot extraction procedure on the plant material of Adenostemma lavenia, and then performing a solid-liquid separation procedure to obtain an extract solution and produce a raffinate of the plant material of Adenostemma lavenia, wherein in the hot extraction procedure, a first 8 ethanol solvent is used as an extraction solvent, and the weight ratio of the first ethanol solvent to the plant material of Adenostemma lavenia is 5-15:1, and the hot extraction procedure is performed at 50-70° C.;
(c) using the raffinate of the plant material of Adenostemma lavenia as the plant material of Adenostemma lavenia, and repeating step (b) at least one time, and combining all the obtained extract solutions to obtain a combined extract solution; and
(d) performing a drying procedure on the combined extract solution to obtain a dry extract.

22. The pharmaceutical composition for treating dermatitis as claimed in claim 21, wherein the extraction method further comprises:
(e) performing a heating procedure on the dry extract in a second ethanol solvent until the dry extract is dissolved to form a mixture, wherein the weight ratio of the second ethanol solvent to the dry extract is 5-15:1, and the heating procedure is performed at 40-60° C.; and
(f) performing column chromatography on the mixture, and collecting, or collecting and combining at least one fraction having an effect of inhibiting cell secretion of IL-4 as an active fraction, wherein in the column chromatography, a mixed solvent of n-hexane/ethyl acetate is used as a mobile phase; and
(g) performing a drying procedure on the active fraction.

23. The pharmaceutical composition for treating dermatitis as claimed in claim 22, wherein the plant material of Adenostemma lavenia is from a leaf of Adenostemma lavenia.

24. The pharmaceutical composition for treating dermatitis as claimed in claim 19, wherein the Adenostemma lavenia extract is a water extract of Adenostemma lavenia.

25. The pharmaceutical composition for treating dermatitis as claimed in claim 24, wherein the water extract of Adenostemma lavenia is obtained by an extraction method, and the extraction method comprises:
(a) providing a plant material of Adenostemma lavenia;
(b) performing a pressurized extraction procedure on the plant material of Adenostemma lavenia, and then performing a solid-liquid separation procedure to obtain an extract solution and produce a raffinate of the plant material of Adenostemma lavenia, wherein in the pressurized extraction procedure, water is used as an extraction solvent, and the weight ratio of the water to the plant material of Adenostemma lavenia is 10-50:1, the pressurized extraction procedure is performed at a pressure of 101.4-140.0 kPa and a temperature of 100-140° C. for 5-60 minutes;
(c) using the raffinate of the plant material of Adenostemma lavenia as the plant material of Adenostemma lavenia, and repeating step (b) at least one time, and combining all the obtained extract solutions to obtain a combined extract solution.
(d) performing a first column chromatography on the combined extract solution, and the first column chromatography comprises:
(d1) filling the combined extract solution into a column packed with resin;
(d2) after step (d1), eluting the column with water, and discarding the eluate that flows out from the column;
(d3) after step (d2), eluting the column with a third ethanol solvent, and discarding the eluate that flows out from the column; and
(d4) after step (d3), eluting the column with a fourth ethanol solvent, and collecting the eluate that flows out from the column as a preliminary purified extract solution;
(e) performing a second column chromatography on the preliminary purified extract solution, and collecting, or collecting and combining at least one fraction having an effect of inhibiting cell secretion of IL-4 as an active fraction, wherein in the second column chromatography, a mixed solvent of n-hexane/ethyl acetate is used as a mobile phase;
(f) concentrating the active fraction to obtain a concentrated active fraction;
(g) performing a heating and dissolving procedure on the concentrated active fraction with a fifth ethanol solvent to form an active solution;
(h) standing the active solution to make crystals form therein; and
(i) collecting the crystals.
